(19) United States
(12) Patent Application Publication
(10) Pub. No.: US 2010/0151056 A1
(43) Pub. Date: Jun. 17, 2010

(54) DIETARY SUPPLEMENTS FOR DECREASING THE LATENT FOREIGN DNA LOAD

(76) Inventor: Hanan Polansky, Rochester, NY (US)

Correspondence Address: Hanan Polansky, 2030 Elmwood Ave., Rochester, NY, 14620, US

(21) Appl. No.: 12/335,576

(22) Filed: Dec. 16, 2008

(51) Int. Cl. A61K 33/04

(52) U.S. Cl. 424/702

(57) ABSTRACT

In one aspect, the invention presents methods for treating chronic diseases. In a preferred embodiment, the methods feature administration to a subject an effective dose of a dietary supplement that prevents or attenuates microcompetition between a foreign polynucleotide and a cellular polynucleotide or attenuates an effect of such microcompetition, especially, when the foreign polynucleotide is latent in the subject.

### I. BACKGROUND OF THE INVENTION

The cause of many cases of the major chronic diseases is unknown. Therefore, treatment is focused on clinical symptoms associated with the disease rather than the cause. As a result, in many cases, the treatment shows limited efficacy and serious negative side effects.

Recently, the National Cancer Institute (NIH Guide 2000¹) announced a program aimed to “reorganize the “front-end,” or gateway, to drug discovery in cancer. The new approach promotes a three stage discovery process; first, discovery of the molecular mechanisms underlying neoplastic transformations, cancer growth and metastasis; second, selection of a novel molecular target within the discovered biochemical pathway associated with the disease state; finally, design of a new drug that modifies the selected target. The program encourages moving away from screening based on a clinical effects, such as tumor cell shrinkage, either in vivo or in vitro, to screening, or drug design, based on molecular effects. According to the NCI, screening by a desired clinical effect identified drugs that traditionally demonstrated clear limitations in patients, while screening by a desired molecular effect should produce more efficacious and specific drugs. ¹ Molecular target drug discovery for cancer: exploratory grants, National Cancer Institute, Feb. 16, 2000, http://grants.nih.gov/grants/guide/pa-files/PAR-00-060.html
² H Polansky. Microcompetition with Foreign DNA and the Origin of Chronic Disease, CBCD Publishing, 543 p, 2003.
³ H Polansky. Assays for drug discovery based on microcompetition with a foreign polynucleotide. U.S. Pat. No. 7,381,526, Jun. 3, 2008.
4 Gonelli A, Boccia S, Boni M, Pozzoli A, Rizzo C, Querzoli P, Cassai E, Di Luca D. Human herpesvirus 7 is latent in gastric mucosa. J Med Virol. April 2007; 63(4):277-83.
⁵ Smith R L, Morroni J, Wilcox C L. Lack of effect of treatment with penciclovir or acyclovir on the establishment of latent HSV-1 in primary sensory neurons in culture. Antiviral Res. October 2001; 52(1):19-24.
⁶ Young L S, Dawson C W, Eliopoulos A G. The expression and function of Epstein-Barr virus encoded latent genes. Mol Pathol. October 2000; 53(5):238-47.
⁷ Virgin H W. Immune regulation of viral infection and vice versa. Immunol Res. 2005; 32(1-3):293-315. Review.
⁸ Gangappa S, Kapadia S B, Speck S H, Virgin H W 4th. Antibody to a lytic cycle viral protein decreases gammaherpesvirus latency in B-cell-deficient mice. J Virol. November 2002; 76(22):11460-8.
⁹ Hoshino Y, Qin J, Follmann D, Cohen J I, Straus S E. The number of herpes simplex virus-infected neurons and the number of viral genome copies per neuron correlate with the latent viral load in ganglia. Virology. Mar. 1, 2008; 372(1):56-63. Epub Nov. 28, 2007.
¹⁰ Kim I J, Flaño E, Woodland D L, Blackman M A. Antibody-mediated control of persistent gammaherpesvirus infection. J Immunol. Apr. 15, 2002; 168(8):3958-64.
¹¹ Kamei Y, Xu L, Heinzel T, Torchia J, Kurokawa R, Gloss B, Lin S C, Heyman R A, Rose D W, Glass C K, Rosenfeld M G. A CBP integrator complex mediates transcriptional activation and AP-1 inhibition by nuclear receptors. Cell. May 3, 1996; 85(3):403-14.
¹² Hottiger M O, Felzien L K, Nabel G J. Modulation of cytokine-induced HIV gene expression by competitive binding of transcription factors to the coactivator p300. EMBO J. Jun. 1, 1998; 17(11):3124-34.
¹³ Kamei Y, Xu L, Heinzel T, Torchia J, Kurokawa R, Gloss B, Lin S C, Heyman R A, Rose D W, Glass C K, Rosenfeld M G. A CBP integrator complex mediates transcriptional activation and AP-1 inhibition by nuclear receptors. Cell. May 3, 1996; 85(3):403-14.
¹⁴ Horvai A E, Xu L, Korzus E, Brard G, Kalafus D, Mullen T M, Rose D W, Rosenfeld M G, Glass C K. Nuclear integration of JAK/STAT and Ras/AP-1 signaling by CBP and p300. Proc Natl Acad Sci U S A. Feb. 18, 1997; 94(4):1074-9.
¹⁵ Hottiger M O, Felzien L K, Nabel G J. Modulation of cytokine-induced HIV gene expression by competitive binding of transcription factors to the coactivator p300. EMBO J. Jun. 1, 1998; 17(11):3124-34.
¹⁶ Pise-Masison C A, Mahieux R, Radonovich M, Jiang H, Brady J N. Human T-lymphotropic virus type I Tax protein utilizes distinct pathways for p53 inhibition that are cell type-dependent. J Biol Chem. Jan. 5, 2001; 276(1):200-5.
¹⁷ Banas B, Eberle J, Banas B, Schlondorff D, Luckow B. Modulation of HIV-1 enhancer activity and virus production by cAMP. FEBS Lett. Dec. 7, 2001; 509(2):207-12.
¹⁸ Wang C, Fu M, D'Amico M, Albanese C, Zhou J N, Brownlee M, Lisanti M P, Chatterjee V K, Lazar M A, Pestell R G. Inhibition of cellular proliferation through IkappaB kinase-independent and peroxisome proliferator-activated receptor gamma-dependent repression of cyclin D1. Mol Cell Biol. May 2001; 21(9):3057-70.
¹⁹ Ernst P, Wang J, Huang M, Goodman R H, Korsmeyer S J. MLL and CREB bind cooperatively to the nuclear coactivator CREB-binding protein. Mol Cell Biol. April 2001; 21(7):2249-58.
²⁰ Yuan W, Varga J. Transforming growth factor-beta repression of matrix metalloproteinase-1 in dermal fibroblasts involves Smad3. J Biol Chem. Oct. 19, 2001; 276(42):38502-10.
²¹ Ghosh A K, Yuan W, Mori Y, Chen Sj, Varga J. Antagonistic regulation of type I collagen gene expression by interferon-gamma and transforming growth factor-beta. Integration at the level of p300/CBP transcriptional coactivators. J Biol Chem. Apr. 6, 2001; 276(14):11041-8.
²² Li M, Pascual G, Glass C K. Peroxisome proliferator-activated receptor gamma-dependent repression of the inducible nitric oxide synthase gene. Mol Cell Biol. July 2000; 20(13):4699-707.
²³ Nagarajan R P, Chen F, Li W, Vig E, Harrington M A, Nakshatri H, Chen Y. Repression of transforming-growth-factor-beta-mediated transcription by nuclear factor kappaB. Biochem J. Jun. 15, 2000; 348 Pt 3:591-6.
²⁴ Speir E, Yu Z X, Takeda K, Ferrans V J, Cannon R O 3rd. Competition for p300 regulates transcription by estrogen receptors and nuclear factor-kappaB in human coronary smooth muscle cells. Circ Res. Nov. 24, 2000; 87(11):1006-11.
²⁵ Chen Y H, Ramos K S. A CCAAT/enhancer-binding protein site within antioxidant/electrophile response element along with CREB-binding protein participate in the negative regulation of rat GST-Ya gene in vascular smooth muscle cells. J Biol Chem. Sep. 1, 2000; 275(35):27366-76.
²⁶ Werner F, Jain M K, Feinberg M W, Sibinga N E, Pellacani A, Wiesel P, Chin M T, Topper J N, Perrella M A, Lee M E. Transforming growth factor-beta 1 inhibition of macrophage activation is mediated via Smad3. J Biol Chem. Nov. 24, 2000; 275(47):36653-8.
²⁷ Nuclear Respiratory Factor 2 should not be confused with NF-E2 Related Factor 2 which is also abbriviated NRF2 or NRF-2.
²⁸ Watanabe H, Imai T, Sharp P A, Handa H. Identification of two transcription factors that bind to specific elements in the promoter of the adenovirus early-region 4. Mol Cell Biol 1988 8(3):1290-300. The transcription factor binds to the promoter of the adenovirus early-region 4 (E4). Hence the name E4 transcription factor 1.
²⁹ Enhancer Factor 1A should not be confused with Elongation Factor 1A which is also abbreviated EF-1A.
³⁰ Suzuki F, Goto M, Sawa C, Ito S, Watanabe H, Sawada J, Handa H. Functional interactions of transcription factor human GA-binding protein subunits. J Biol Chem. Nov. 6, 1998; 273(45):29302-8.
³¹ Asano M, Murakami Y, Furukawa K, Yamaguchi-Iwai Y, Stake M, Ito Y. A Polayomavirus Enhancers Binding Protein, PEBP5, Responsive to 12-O-Tetradecanoylphorbol-13-Acetate but Distinct From AP-1. Journal of Virology 1990 64(12):5927-5938.
³² Higashino F, Yoshida K, Fujinaga Y, Kamio K, Fujinaga K. Isolation fo a cDNA Encoding the Adenovirus E1A Enhancer Binding Protein: A New Human Member of the ets Oncogene Family. Nucleic Acids Research 1993 21(3):547-553.
³³ Laimins L A, Tsichlis P, Khoury G. Multiple Enhancer Domains in the 3′ Terminus of the Prague Strain of Rous Sarcoma Virus. Nucleic Acids Research 1984 12(16):6427-6442.
³⁴ LaMarco K L, McKnight S L. Purification of a set of cellular polypeptides that bind to the purine-rich cis-regulatory element of herpes simplex virus immediate early genes. Genes Dev 1989 3(9):1372-83.
³⁵ Douville P, Hagmann M, Georgiev O, Schaffner W. Positive and negative regulation at the herpes simplex virus ICP4 and ICP0 TAATGARAT motifs. Virology. Feb. 20, 1995; 207(1):107-16.
³⁶ Boshart M, Weber F, Jahn G, Dorsch-Hasler K, Fleckenstein B, Schaffner W. A very strong enhancer is located upstream of an immediate early gene of human cytomegalovirus. Cell June 1985; 41(2):521-30.
³⁷ Gunther C V, Graves B J. Identification of ETS domain proteins in murine T lymphocytes that interact with the Moloney murine leukemia virus enhancer. Mol Cell Biol 1994 14(11): 7569-80
³⁸ Flory E, Hoffmeyer A, Smola U, Rapp U R, Bruder J T. Raf-1 kinase targets GA-binding protein in transcriptional regulation of the human immunodeficiency virus type 1 promoter. J Virol April 1996; 70(4):2260-8.
³⁹ Rawlins D R, Milman G, Hayward S D, Hayward G S. Sequence-specific DNA binding of the Epstein-Barr virus nuclear antigen (EBNA-1) to clustered sites in the plasmid maintenance region. Cell October 1985; 42(3):859-68.
⁴⁰ Mauclere P, Mahieux R, Garcia-Calleja J M, Salla R, Tekaia F, Millan J, De The G, Gessain A. A new HTLV-II subtype A isolate in an HIV-1 infected prostitute from Cameroon, Central Africa. AIDS Res Hum Retroviruses. August 1995; 11(8):989-93.
⁴¹ Komfeld H, Riedel N, Viglianti G A, Hirsch V, Mullins J I. Cloning of HTLV-4 and its relation to simian and human immunodeficiency viruses. Nature 1987 326(6113);610-613.
⁴² Bannert N, Avots A, Baier M, Serfling E, Kurth R. GA-binding protein factors, in concert with the coactivator CREB binding protein/p300, control the induction of the interleukin 16 promoter in T lymphocytes. Proc, Natl, Acad, Sci, USA 1999 96:1541-1546.
⁴³ Flory E, Hoffmeyer A, Smola U, Rapp U R, Bruder J T. Raf-1 kinase targets GA-binding protein in transcriptional regulation of the human immunodeficiency virus type 1 promoter. J Virol April 1996; 70(4):2260-8.
⁴⁴ Douville P, Hagmann M, Georgiev O, Schaffner W. Positive and negative regulation at the herpes simplex virus ICP4 and ICP0 TAATGARAT motifs. Virology. Feb. 20, 1995; 207(1):107-16.
⁴⁵ Bruder J T, Hearing P. Cooperative binding of EF-1A to the E1A enhancer region mediates synergistic effects on E1A transcription during adenovirus infection. J Virol. September 1991; 65(9):5084-7.
⁴⁶ Bruder J T, Hearing P. Nuclear factor EF-1A binds to the adenovirus ElA core enhancer element and to other transcriptional control regions. Mol Cell Biol. November 1989; 9(11):5143-53.
⁴⁷ Ostapchuk P, Diffley J F, Bruder J T, Stillman B, Levine A J, Hearing P. Interaction of a nuclear factor with the polyomavirus enhancer region. Proc Natl Acad Sci U S A. November 1986; 83(22):8550-4.
⁴⁸ Gunther C V, Graves B J. Identification of ETS domain proteins in murine T lymphocytes that interact with the Moloney murine leukemia virus enhancer. Mol Cell Biol 1994 14(11): 7569-80
⁴⁹ Scholer H R, Gruss P. Specific interaction between enhancer-containing molecules and cellular components. Cell. February 1984; 36(2):403-11.
⁵⁰ Chen L F, Ito K, Murakami Y, Ito Y. The capacity of polyomavirus enhancer binding protein 2alphaB (AML1/Cbfa2) to stimulate polyomavirus DNA replication is related to its affinity for the nuclear matrix. Mol Cell Biol. July 1998; 18(7):4165-76.
⁵¹ Lewis A F, Stacy T, Green W R, Taddesse-Heath L, Hartley J W, Speck N A. Core-binding factor influences the disease specificity of Moloney murine leukemia virus. J Virol. July 1999; 73(7):5535-47.
⁵² Sun W, Graves B J, Speck N A. Transactivation of the Moloney murine leukemia virus and T-cell receptor beta-chain enhancers by cbf and ets requires intact binding sites for both proteins. J Virol. August 1995; 69(8):4941-9.
⁵³ Martiney M J, Rulli K, Beaty R, Levy L S, Lenz J. Selection of reversions and suppressors of a mutation in the CBF binding site of a lymphomagenic retrovirus. J Virol. September 1999; 73(9):7599-606.
⁵⁴ Martiney M J, Levy L S, Lenz J. Suppressor mutations within the core binding factor (CBF/AML1) binding site of a T-cell lymphomagenic retrovirus. J Virol. March 1999; 73(3):2143-52.
⁵⁵ Cron R Q, Bartz S R, Clausell A, Bort S J, Klebanoff S J, Lewis D B. NFAT1 enhances HIV-1 gene expression in primary human CD4 T cells. Clin Immunol. March 2000; 94(3):179-91.
⁵⁶ Wang W X, Li M, Wu X, Wang Y, Li Z P. HNF1 is critical for the liver-specific function of HBV enhancer II. Res Virol. March-April 1998; 149(2):99-108.
⁵⁷ Liang C L, Tsai C N, Chung P J, Chen J L, Sun C M, Chen R H, Hong J H, Chang Y S. Transcription of Epstein-Barr virus-encoded nuclear antigen 1 promoter Qp is repressed by transforming growth factor-beta via Smad4 binding element inhuman BL cells. Virology. Nov. 10, 2000; 277(1):184-92.
⁵⁸ Chen J, Stinski M F. Activation of transcription of the human cytomegalovirus early UL4 promoter by the Ets transcription factor binding element. J Virol. November 2000; 74(21):9845-57.
⁵⁹ Huang L, Malone C L, Stinski M F. A human cytomegalovirus early promoter with upstream negative and positive cis-acting elements: IE2 negates the effect of the negative element, and NF-Y binds to the positive element.
⁶⁰ Lu C C, Yen T S. Activation of the hepatitis B virus S promoter by transcription factor NF-Y via a CCAAT element. Virology. Nov. 15, 1996; 225(2):387-94.
⁶¹ Bock C T, Kubicka S, Manns M P, Trautwein C. Two control elements in the hepatitis B virus S-promoter are important for full promoter activity mediated by CCAAT-binding factor. Hepatology. April 1999; 29(4):1236-47.
⁶² Gu Z, Plaza S, Perros M, Cziepluch C, Rommelaere J, Cornelis J J. NF-Y controls transcription of the minute virus of mice P4 promoter through interaction with an unusual binding site. J Virol. January 1995; 69(1):239-46.
⁶³ Song B, Young CS. Functional analysis of the CAAT box in the major late promoter of the subgroup C human adenoviruses. J Virol. April 1998; 72(4):3213-20.
⁶⁴ Moriuchi H, Moriuchi M, Cohen J I. The varicella-zoster virus immediate-early 62 promoter contains a negative regulatory element that binds transcriptional factor NF-Y. Virology. Dec. 1, 1995; 214(1):256-8.
⁶⁵ Xu X, Kang S H, Heidenreich O, Brown D A, Nerenberg M I. Sequence requirements of ATF2 and CREB binding to the human T-cell leukemia virus type 1 LTR R region. Virology. Apr. 15, 1996; 218(2):362-71.
⁶⁶ Xu X, Brown D A, Kitajima I, Bilakovics J, Fey L W, Nerenberg M I. Transcriptional suppression of the human T-cell leukemia virus type I long terminal repeat occurs by an unconventional interaction of a CREB factor with the R region. Mol Cell Biol. August 1994; 14(8):5371-83.
⁶⁷ Choi C Y, Choi B H, Park G T, Rho H M. Activating transcription factor 2 (ATF2) down-regulates hepatitis B virus X promoter activity by the competition for the activating protein 1 binding site and the formation of the ATF2-Jun heterodimer. J Biol Chem. Jul. 4, 1997; 272(27):16934-9.
⁶⁸ Kanda T, Segawa K, Ohuchi N, Mori S, Ito Y. Stimulation of polyomavirus DNA replication by wild-type p53 through the DNA-binding site. Mol Cell Biol. April 1994; 14(4):2651-63.
⁶⁹ Allamane S, Ratel D, Jourdes P, Berger F, Benabid A L, Wion D. p53 Status and gene transfer experiments using CMV enhancer/promoter. Biochem Biophys Res Commun. Jan. 12, 2001; 280(1):45-7.
⁷⁰ Deb D, Lanyi A, Scian M, Keiger J, Brown D R, Le Roith D, Deb S P, Deb S. Differential modulation of cellular and viral promoters by p73 and p53. Int J Oncol. February 2001; 18(2):401-9.
⁷¹ Lee H, Kim H T, Yun Y. Liver-specific enhancer II is the target for the p53-mediated inhibition of hepatitis B viral gene expression. J Biol Chem. Jul. 31, 1998; 273(31):19786-91.
⁷² Ori A, Zauberman A, Doitsh G, Paran N, Oren M, Shaul Y. p53 binds and represses the HBV enhancer: an adjacent enhancer element can reverse the transcription effect of p53. EMBO J. Jan. 15, 1998; 17(2):544-53.
⁷³ Jundt F, Herr I, Angel P, Zur Hausen H, Bauknecht T. Transcriptional control of human papillomavirus type 18 oncogene expression in different cell lines: role of transcription factor YY1. Virus Genes. 1995; 11(1):53-8.
⁷⁴ Lai C K, Ting L P. Transcriptional repression of human hepatitis B virus genes by a bZIP family member, E4BP4. J Virol. April 1999; 73(4):3197-209.
⁷⁵ Gilbert S, Galameau L, Lamontagne A, Roy S, Belanger L. The hepatitis B virus core promoter is strongly activated by the liver nuclear receptor fetoprotein transcription factor or by ectopically expressed steroidogenic factor 1. J Virol. June 2000; 74(11):5032-9.
⁷⁶ Honda Y, Rogers L, Nakata K, Zhao B Y, Pine R, Nakai Y, Kurosu K, Rom W N, Weiden M. Type I interferon induces inhibitory 16-kD CCAAT/enhancer binding protein (C/EBP)beta, repressing the HIV-1long terminal repeat in macrophages: pulmonary tuberculosis alters C/EBP expression, enhancing HIV-1 replication. J Exp Med. Oct. 5, 1998; 188(7):1255-65.
⁷⁷ Bannert N, Avots A, Baier M, Serfling E, Kurth R. GA-binding protein factors, in concert with the coactivator CREB binding protein/p300, control the induction of the interleukin 16 promoter in T lymphocytes. Proc, Natl, Acad, Sci, USA 1999 96:1541-1546.
⁷⁸ Kitabayashi I, Yokoyama A, Shimizu K, Ohki M. Interaction and functional cooperation of the leukemia-associated factors AML1 and p300 in myeloid cell differentiation. EMBO J. Jun. 1, 1998; 17(11):2994-3004.
⁷⁹ Garcia-Rodriguez C, Rao A. Nuclear factor of activated T cells (NFAT)-dependent transactivation regulated by the coactivators p300/CREB-binding protein (CBP). J Exp Med. Jun. 15, 1998; 187(12):2031-6.
⁸⁰ Sisk T J, Gourley T, Roys S, Chang C H. MHC class II transactivator inhibits IL-4 gene transcription by competing with NF-AT to bind the coactivator CREB binding protein (CBP)/p300. J Immunol. Sep. 1, 2000; 165(5):2511-7.
⁸¹ Soutoglou E, Papafotiou G, Katrakili N, Talianidis I. Transcriptional activation by hepatocyte nuclear factor-1 requires synergism between multiple coactivator proteins. J Biol Chem. Apr. 28, 2000; 275(17):12515-20.
⁸² Janknecht R, Wells N J, Hunter T. TGF-beta-stimulated cooperation of smad proteins with the coactivators CBP/p300. Genes Dev. Jul. 15, 1998; 12(14):2114-9.
⁸³ Feng X H, Zhang Y, Wu R Y, Derynck R. The tumor suppressor Smad4/DPC4 and transcriptional adaptor CBP/p300 are coactivators for smad3 in TGF-beta-induced transcriptional activation. Genes Dev. Jul. 15, 1998; 12(14):2153-63.
⁸⁴ Pouponnot C, Jayaraman L, Massague J. Physical and functional interaction of SMADs and p300/CBP. J Biol Chem. Sep. 4, 1998; 273(36):22865-8.
⁸⁵ Jayaraman G, Srinivas R, Duggan C, Ferreira E, Swaminathan S, Somasundaram K, Williams J, Hauser C, Kurkinen M, Dhar R, Weitzman S, Buttice G, Thimmapaya B. p300/cAMP-responsive element-binding protein interactions with ets-1 and ets-2 in the transcriptional activation of the human stromelysin promoter. J Biol Chem. Jun. 11, 1999; 274(24):17342-52.
⁸⁶ Li Q, Herrler M, Landsberger N, Kaludov N, Ogryzko V V, Nakatani Y, Wolffe A P. Xenopus NF-Y pre-sets chromatin to potentiate p300 and acetylation-responsive transcription from the Xenopus hsp70 promoter in vivo. EMBO J. Nov. 2, 1998; 17(21):6300-15.
⁸⁷ Duyndam M C, van Dam H, Smits P H, Verlaan M, van der Eb A J, Zantema A. The N-terminal transactivation domain of ATF2 is a target for the co-operative activation of the c-jun promoter by p300 and 12S EL1A. Oncogene. Apr. 8, 1999; 18(14):2311-21.
⁸⁸ Avantaggiati M L, Ogryzko V, Gardner K, Giordano A, Levine A S, Kelly K. Recruitment of p300/CBP in p53-dependent signal pathways. Cell. Jun. 27, 1997; 89(7):1175-84.
⁸⁹ Van Orden K, Giebler HA, Lemasson I, Gonzales M, Nyborg J K. Binding of p53 to the KIX domain of CREB binding protein. A potential link to human T-cell leukemia virus, type I-associated leukemogenesis. J Biol Chem. Sep. 10, 1999; 274(37):26321-8.
⁹⁰ Hottiger M O, Felzien L K, Nabel G J. Modulation of cytokine-induced HIV gene expression by competitive binding of transcription factors to the coactivator p300. EMBO J. Jun. 1, 1998; 17(11):3124-34.
⁹¹ Gerritsen M E, Williams A J, Neish A S, Moore S, Shi Y, Collins T. CREB-binding protein/p300 are transcriptional coactivators of p65. Proc Natl Acad Sci USA. Apr. 1, 1997; 94(7):2927-32.
⁹² Bhattacharya S, Eckner R, Grossman S, Oldread E, Arany Z, D'Andrea A, Livingston D M. Cooperation of Stat2 and p300/CBP in signalling induced by interferon-alpha. Nature. Sep. 26, 1996; 383(6598):344-7.
⁹³ Mink S, Haenig B, Klempnauer K H. Interaction and functional collaboration of p300 and C/EBPbeta. Mol Cell Biol. November 1997; 17(11):6609-17.
⁹⁴ Beattie J H, Wood A M, Newman A M, Bremner I, Choo K H A, Michalska A E, Duncan J S, Trayhurn P. Obesity and hyperleptinemia in metallothionein (-I and -II) null mice. Proc. Natl. Acad. Sci. USA 1998 95(1):358-363.
⁹⁵ Qu W, Diwan B A, Liu J, Goyer R A, Dawson T, Horton J L, Cherian M G, Waalkes M P. The metallothionein-null phenotype is associated with heightened sensitivity to lead toxicity and an inability to form inclusion bodies. Am J Pathol. March 2002; 160(3):1047-56.
⁹⁶ Penkowa M, Espejo C, Martinez-Caceres E M, Poulsen C B, Montalban X, Hidalgo J. Altered inflammatory response and increased neurodegeneration in metallothionein I+II deficient mice during experimental autoimmune encephalomyelitis. J Neuroimmunol. Oct. 1, 2001; 119(2):248-60.
⁹⁷ Reeve V E, Nishimura N, Bosnic M, Michalska A E, Choo K H. Lack of metallothionein-I and -II exacerbates the immunosuppressive effect of ultraviolet B radiation and cis-urocanic acid in mice. Immunology. July, 2000; 100(3):399-404.
⁹⁸ Liu J, Liu Y, Goyer R A, Achanzar W, Waalkes M P. Metallothionein-I/II null mice are more sensitive than wild-type mice to the hepatotoxic and nephrotoxic effects of chronic oral or injected inorganic arsenicals. Toxicol Sci. June 2000; 55(2):460-7.
⁹⁹ Kaminski W E, Lindahl P, Lin N L, Broudy V C, Crosby J R, Hellstrom M, Swolin B, Bowen-Pope D F, Martin P J, Ross R, Betsholtz C, Raines E W. Basis of hematopoietic defects in platelet-derived growth factor (PDGF)-B and PDGF beta-receptor null mice. Blood. Apr. 1, 2001; 97(7):1990-8.
¹⁰⁰ Lindahl P, Johansson B R, Leveen P, Betsholtz C. Pericyte loss and microaneurysm formation in PDGF-B-deficient mice. Science. Jul. 11, 1997; 277(5323):242-5.
¹⁰¹ Osuga J, Ishibashi S, Oka T, Yagyu H, Tozawa R, Fujimoto A, Shionoiri F, Yahagi N, Kraemer F B, Tsutsumi O, Yamada N. Targeted disruption of hormone-sensitive lipase results in male sterility and adipocyte hypertrophy, but not in obesity. Proc Natl Acad Sci USA. Jan. 18, 2000; 97(2):787-92.
¹⁰² Roduit R, Masiello P, Wang S P, Li H, Mitchell G A, Prentki M. A role for hormone-sensitive lipase in glucose-stimulated insulin secretion: a study in hormone-sensitive lipase-deficient mice. Diabetes. September 2001; 50(9):1970-5.
¹⁰³ Chung S, Wang S P, Pan L, Mitchell G, Trasler J, Hermo L. Infertility and testicular defects in hormone-sensitive lipase-deficient mice. Endocrinology. October 2001; 142(10):4272-81.
¹⁰⁴ Haemmerle G, Zimmermann R, Strauss J G, Kratky D, Riederer M, Knipping G, Zechner R. Hormone-sensitive lipase deficiency in mice changes the plasma lipid profile by affecting the tissue-specific expression pattern of lipoprotein lipase in adipose tissue and muscle. J Biol Chem. Apr. 12, 2002; 277(15):12946-52.
¹⁰⁵ Haemmerle G, Zimmermann R, Hayn M, Theussl C, Waeg G, Wagner E, Sattler W, Magin T M, Wagner E F, Zechner R. Hormone-sensitive lipase deficiency in mice causes diglyceride accumulation in adipose tissue, muscle, and testis. J Biol Chem. Feb. 15, 2002; 277(7):4806-15.
¹⁰⁶ Curreli F, Alvin E. Friedman-Kien A E, Flore O. Glycyrrhizic acid alters Kaposi sarcoma associated herpesvirus latency, triggering p53-mediated apoptosis in transformed B lymphocytes. J Clin Invest. Mar. 1, 2005; 115(3):642-652.
¹⁰⁷ Babcock G J, Decker L L, Freeman R B, Thorley-Lawson D A. Epstein-barr virus-infected resting memory B cells, not proliferating lymphoblasts, accumulate in the peripheral blood of immunosuppressed patients. J Exp Med. Aug. 16, 1999; 190(4):567-76.
¹⁰⁸ Lin J C, Chemg J M, Hung M S, Baltina L A, Baltina L, Kondratenko R. Inhibitory effects of some derivatives of glycyrrhizic acid against Epstein-Barr virus infection: structure-activity relationships. Antiviral Res. July 2008; 79(1):6-11. Epub Mar. 31, 2008.
¹⁰⁹ Prang N S, Homef M W, Jager M, Wagner H J, Wolf H, Schwarzmann F M. Lytic replication of Epstein-Barr virus in the peripheral blood: analysis of viral gene expression in B lymphocytes during infectious mononucleosis and in the normal carrier state. Blood. Mar. 1, 1997; 89(5):1665-77.
¹¹⁰ Adams A. Replication of latent Epstein-Barr virus genomes in Raji cells. J Virol. May 1987; 61(5): 1743-1746.
¹¹¹ Iwase Y, Takemura Y, Ju-ichi M, Mukainaka T, Ichiishi E, Ito C, Furukawa H, Yano M, Tokuda H, Nishino H. Inhibitory effect of flavonoid derivatives on Epstein-Barr virus activation and two-stage carcinogenesis of skin tumors. Cancer Lett. Nov. 28, 2001; 173(2):105-9.
¹¹² Wu L L, Yang X B, Huang Z M, Liu H Z, Wu G X. In vivo and in vitro antiviral activity of hyperoside extracted from Abelmoschus manihot (L) medik. Acta Pharmacol Sin. March 2007; 28(3):404-9.
¹¹³ Favre D, Petit M A, Trépo C. Latent hepatitis B virus (HBV) infection and HBV DNA integration is associated with further transformation of hepatoma cells in vitro. ALTEX. 2003; 20(3):131-42.
¹¹⁴ Laras A, Koskinas J, Dimou E, Kostamena A, Hadziyannis S J. Intrahepatic levels and replicative activity of covalently closed circular hepatitis B virus DNA in chronically infected patients. Hepatology. September 2006; 44(3):694-702.
¹¹⁵ Chang L K, Wei T T, Chiu Y F, Tung C P, Chuang J Y, Hung S K, Li C, Liu S T. Inhibition of Epstein-Barr virus lytic cycle by (−)-epigallocatechin gallate. Biochem Biophys Res Commun. Feb. 21, 2003; 301(4):1062-8.
¹¹⁶ Pavlova I V, Virgin H W 4th, Speck S H. Disruption of gammaherpesvirus 68 gene 50 demonstrates that Rta is essential for virus replication. J Virol. May 2003; 77(10):5731-9
¹¹⁷ Xu J, Wang J, Deng F, Hu Z, Wang H. Green tea extract and its major component epigallocatechin gallate inhibits hepatitis B virus in vitro. Antiviral Res. June 2008; 78(3):242-9. Epub Feb. 8, 2008.
¹¹⁸ Motohashi N, Yamagami C, Tokuda H, Okuda Y, Ichiishi E, Mukainaka T, Nishino H, Saito Y. Structure-activity relationship in potentially anti-tumor promoting benzalacetone derivatives, as assayed by the epstein-barr virus early antigen activation. Mutat Res. Jan. 24, 2000; 464(2):247-54.
¹¹⁹ Jian S W, Mei C E, Liang Y N, Li D, Chen Q L, Luo H L, Li Y Q, Cai T Y. [Influence of selenium-rich rice on transformation of umbilical blood B lymphocytes by Epstein-Barr virus and Epstein-Barr virus early antigen expression]. Ai Zheng. January 2003; 22(1):26-9. [Article in Chinese]
¹²⁰ Shapira M Y, Resnick I B, Chou S, Neumann A U, Lurain N S, Stamminger T, Caplan O, Saleh N, Efferth T, Marschall M, Wolf D G. Artesunate as a potent antiviral agent in a patient with late drug-resistant cytomegalovirus infection after hematopoietic stem cell transplantation. Clin Infect Dis. May 1, 2008; 46(9):1455-7.
¹²¹ Schroeder R, Michelon T, Fagundes I, Bortolotto A, Lammerhirt E, Oliveira J, Santos A, Bittar A, Keitel E, Garcia V, Neumann J, Saitovitch D. Cytomegalovirus disease latent and active infection rates during the first trimester after kidney transplantation. Transplant Proc. May 2004; 36(4):896-8.
¹²² Emery V C, Cope A V, Bowen E F, Gor D, Griffiths P D. The dynamics of human cytomegalovirus replication in vivo. J Exp Med 1999; 190: 177-82.

The best drugs reverse the molecular events that cause a disease. Following the discovery of microcompetition between foreign polynucleoitdes and cellular genes as the cause of many chronic disease cases, see (Polansky 2003²) and U.S. Pat. No. 7,381,526³, hereby expressly and entirely incorporated by reference, the present invention presents methods for preventing and treating chronic diseases using dietary supplements.

### II. BRIEF SUMMARY OF THE INVENTION

In one aspect, the invention presents methods for treating chronic diseases. In a preferred embodiment, the methods feature administration to a subject an effective dose of a dietary supplement that prevents or attenuates microcompetition between a foreign polynucleotide and a cellular polynucleotide or attenuates an effect of such microcompetition, especially, when the foreign polynucleotide is latent in the subject.

For example, to ameliorate a disease symptom resulting from microcompetition between a foreign polynucleotide and a cellular polynucleotide, a dietary supplement can be administered to the subject to reduce the cellular copy number of the foreign polynucleotide, to reduce the formation of a complex between the foreign polynucleotide and a cellular transcription factor, to increase the formation of a complex between the microcompeted cellular transcription factor and the cellular polynucleotide, or to reverse an effect of microcompetition on the expression or activity of a polypeptide with expression regulated by the cellular polynucleotide, directly, or indirectly through, for instance, increasing the effectiveness of the immune system, or modifying a function of the immune system.

### III. DETAILED DESCRIPTION OF THE INVENTION

The following sections present descriptions of elements used in the present invention. Following each definition, one or more exemplary assays are provided to illustrate to one skilled in the art how to use the element. Each assay may include, as its own elements, standard methods in molecular biology, microbiology, cell biology, cell culture, transgenic biology, recombinant DNA, immunology, pharmacology, and toxicology, well known in the art.

A. Foreign Polynucleotides and Aberrant Transcription

1. Foreign Polynucleotides

Definition 1

Consider an organism with a certain standard genome. If a polynucleotide is different from all segments of the standard genome, the polynucleotide will be called “foreign to” the organism.

Notes:

1. As an example for different organisms consider the list of standard organisms in the Patentln 3.1 software. The list includes organisms such as, homo sapiens (human), mus musculus (mouse), ovis aries (sheep), and gallus gallus (chicken).

2. A standard genome is the genome shared by most representatives of the same organism.

3. A polynucleotide and DNA sequence (see above) are interchangeable concepts.

4. In multicellular organism, such as humans, the standard genome of the organism is not necessarily found in every cell. The genomes found in sampled cells can vary as a result of somatic mutations, viral integration, etc. (see definition below of foreign polynucleotide in a specific cell).

5. Assume the polynucleotide expresses a polypeptide. If the polynucleotide is foreign to the organism, then its polypeptide is also foreign to the organism.

6. When the reference organism is evident, instead of the phrase “a polynucleotide foreign to organism X,” the “foreign polynucleotide” phrase might be used.

Exemplary Assays

1. Compare the sequence of the polynucleotide with the sequence, or sequences of the published, or self sequenced standard genome of the organism. If the sequence is not a segment of the standard genome, the polynucleotide is foreign to the organism.

2. Isolate DNA from the organism genome (for instance, from a specific cell, or a virus). Try to hybridize the polynucleotide of interest to the isolated DNA. If the polynucleotide of interest does not hybridize, it is foreign.

Notes:

1. A polynucleotide can still be foreign if it hybridizes with DNA from a specific segment of the genome. Consider, for example, the case of integrated viral genomes. Viral sequences integrated into cellular genomes are foreign. To increase the probability of correct identification, repeat the assay with many specimens of the genome (for instance, by collecting many cells from different representatives of the organism). Define the genome of the organism as all DNA sequences found in all isolated specimens. Following this definition, integrated sequences which are only segments of certain specimens are identified as foreign. Note that the test is dependent on the size of the population. For instance, a colony which propagates from a single cell might include a foreign polynucleotide in all daughter cells. Therefore, the specimens should include genomes (or cells) from different lineages.

2. A polynucleotide can also be identified as potentially foreign if it is found episomally in the nucleus. If the DNA is found in the cytoplasm, it is most likely foreign. Also, a large enough polynucleotide can be identified as foreign if many copies of the polynucleotide can be observed in the nucleus. Finally, if the polynucleotide is identical to sequences in genomes of other organisms, such as viruses or bacteria, known to invade the organisms cells, and specifically nuclei of the organisms cells, the polynucleotide is likely foreign to organism.

Definition 2

Consider an organism. If a polynucleotide of interest is immunologically foreign to the organism, the polynucleotide is called “foreign to the organism.”

Notes:

1. In Definition 1, the comparison between the genome of the organism and the polynucleotide is performed logically by the observer. In definition 2, the comparison is performed biologically by the immune system of the organism.

2. Definition 2 can be generalized to any compound or substance. A certain compound is called foreign to a certain organism, if the compound is immunologically foreign to the organism.

Exemplary Assays

1. If the test polynucleotide includes a coding region, incorporate the test polynucleotide in an expressing plasmid and transfer the plasmid into the organism, through, for instance, injection. An immune response against the expressed polypeptide indicates that the polynucleotide is foreign.

2. Inject the test polynucleotide in the organism. An immune response against the injected polynucleotide indicates that the test polynucleotide is foreign.

Examples

Many viruses, nuclear, such as Epstein-Barr, and cytoplasmic, such as Vaccinia, express proteins which are antigenic and immunogenic in their respective host cells.

Definition 3

Consider an organism with a certain standard genome. Consider a segment of the genome. If a polynucleotide of interest is chemically or physically different from all possible segments of the genome, the polynucleotide will be called “foreign to the organism.”

Notes:

1. In Definition 3, the observer compares the genome of the organism with the polynucleotide using the chemical or physical characteristics of the molecules.

Exemplary Assays

In general, many assays compare a test polynucleotide and a wild-type polynucleotide. These assays determine if the test polynucleotide is foreign to the wild-type polynucleotide in the following exemplary ways:

1. Comparing the electrophoretic gel mobility of the two polynucleotides. If mobility is different, the polynucleotides are different.

2. Comparing the patterns of restriction enzyme cleavage of the two polynucleotides. If the patterns are different, the polynucleotides are different.

3. Comparing the patterns of methylation the two polynucleotides (by, for instance, electrophoretic gel mobility). If the patterns are different, the polynucleotides are different.

Definition 4

Consider an organism with standard genome. Consider a cell. If the copy number of the polynucleotide of interest in the cell is different than the copy number of the polynucleotide in the genome, the polynucleotide is called “foreign to the cell.”

Notes:

1. By “the copy number of the polynucleotide in the cell” we mean the copy number of the polynucleotide from all sources in the cell. For instance, the copy number includes all the polynucleotide segments in the genome, all the polynucleotide segments of viral DNA in the cell (if available), all the polynucleotide segments of plasmid DNA in the cell (if available), etc.

2. If copy number of the polynucleotide in zero, the definition is identical to definition 1 of foreign polynucleotide.

Exemplary Assays

1. Sequence the genome of the cell. Count the number of time the polynucleotide appears in the genome. Compare the result to the number of times the polynucleotide appears in the published standard genome. If the number is greater, the polynucleotide is foreign to the cell.

2. Sequence the genome of the cell and a group of other cells from the same organism. If the copy number is different in the given cell, the polynucleotide is foreign to the given cell.

See more explanations and examples in U.S. Pat. No. 7,381,526 (ibid), hereby expressly and entirely incorporated by reference.

2. Latent and Persistent Foreign Polynucleotides

Definition

Consider a polynucleotide foreign to a given organism. The polynucleotide will be called latent in a cell of the organism if over an extended period of time, either:

1. The polynucleotide produces no transcripts.

2. The set of gene products expressed by the polynucleotide in the cell is a subset of all possible gene products of that polynucleotide.

3. The polynucleotide shows limited or no replication.

4. The polynucleotide is undetected by the host immune system.

5. The shows no lytic symptoms.

6. The organism shows no macroscopic symptoms.

Notes:

1. A virus in a host cell is a foreign polynucleotide. According to the definition, a virus is considered latent if, over an extended period of time, it either shows partial expression of its gene products, no viral mRNA, limited or no replication, is undetected by the host immune system, causes no lytic symptoms in the infected cell, or causes no macroscopic symptoms in the host.

2. The above list of characterizations is not exhaustive. The medical literature includes more aspects of latency that can be added to the definition.

Exemplary Assays

1. Introduce, or identify a foreign polynucleotide in a host cell. Assay the polynucleotide replication, or transcription, or mRNA, or gene products over an extended period of time. If the polynucleotide shows limited replication, no transcription, or a limited set of transcripts, the polynucleotide is latent.

2. Introduce, or identify a foreign polynucleotide in a host cell. Assay the cell over an extended period of time, if the cell shows no lytic symptoms, the polynucleotide is latent.

Examples

Using PCR, a study (Gonelli 20014) observed persistent presence of viral human herpes virus 7 (HHV-7) DNA in biopsies from 50 patients with chronic gastritis. The study also observed no U14, U17/17, U31, U42 and U89/90, HHV-7 specific transcripts highly expressed during replication. Based on these observations, the study concluded that “gastric tissue represents a site of HHV-7 latent infection and potential reservoir for viral reactivation.” To test the effect of treatment on the establishment of latent herpes simplex virus, type 1 (HSV-1) in sensory neurons, another study (Smith 20015) assays the expression of the latency-associated transcript (LAT), the only region of the viral genome transcribed at high levels during the period of viral latency. A recent review (Young 20006) discusses the limited sets of Epstein-Barr viral (EBV) gene products expressed during the period of viral latency.

See more explanations and examples in U.S. Pat. No. 7,381,526 (ibid).

Each individual host has a latency set point, or small range, at which the immune system and viral factors that control the level of latency are in equilibrium (Virgin 2005⁷). According to Virgin 2005 (ibid), “There are good data that persistent replication occurs in both normal and immunocompromised mice and that it can contribute to the pool of latently infected cells.” Also, according to Virgin 2005 (ibid), “In addition, persistent replication can significantly contribute to maintenance of a pool of latently infected cells, perhaps via infection of new cells in which the virus can establish latency. For example, in B-cell-deficient mice both persistent replication and increased number of latently infected cells are observed. If these mice are treated with either an antiviral Ab specific for a lytic cycle viral antigen or an antiviral drug that targets the viral DNA polymerase, the number of latently infected cells diminishes dramatically. This is most consistent with the role for persistent γHV68 replication as a major contributor to the size of the latently infected cell pool.” Also consider Gangappa 2002⁸: “In the experiments reported here, we determined the effect of passively transferred antibody on established γHV68 latency in B-cell-deficient (B-cell(−/−)) mice. Immune antibody decreased the frequency of cells reactivating ex vivo from latency in splenocytes (>10-fold) and peritoneal cells (>100-fold) and the frequency of cells carrying latent viral genome in splenocytes (>5-fold) and peritoneal cells (>50-fold). . . . Passive transfer of antibody specific for the lytic cycle γHV68 RCA protein decreased both the frequency of cells reactivating ex vivo from latency and the frequency of cells carrying the latent viral genome. Therefore, antibody specific for lytic cycle viral antigens can play an important role in the control of gammaherpesvirus latency in immunocompromised hosts. Based on these findings, we propose a model in which ongoing productive replication is essential for maintaining high levels of latently infected cells in immunocompromised hosts. We confirmed this model by the treatment of latently infected B-cell(−/−) mice with the antiviral drug cidofovir.” Also “antibody to lytic cycle can decrease the frequency of latently infected cells by breaking a cycle in which lytic replication contributes to the pool of latently infected cells in B-cell(−/−) mice.” . . . “This in turn suggests that ongoing or sporadic productive infection plays an important part in the maintenance of high frequencies of latently infected cells in B-cell(−/−) mice. Consistent with this model, inhibition of productive infection with an antiviral drug also decreased the frequency of latently infected cells.” See also Virgin 2005 (ibid): “Finally, there is excellent evidence that the immune system is important for maintaining a normal level of latently infected cells and for regulating the efficiency of reactivation from latency. In addition, the frequency of cells latently infected with EBV is significantly increased in immunocompromised patients.” See also Hosino 2008⁹ and Kim 2002¹⁰.

These studies suggest that any disruption of persistent replication or reactivation from latency diminishes the number of latently infected cells or the virus copy number in latently infected cells, together defining the latent viral load.

Also, according to Virgin 2005 (ibid): “Several groups have tested the hypothesis that high levels of preexisting immunity might attenuate chronic γ-herpesvirus disease by limiting infection. Vaccination against γHV68 infection with single viral antigens attenuates acute infection and decreases the amount of latent infection at early time points (2 to 3 wk infection). For example, vaccination against the major membrane glycoprotein gp150 induced a neutralizing Ab response and reduced the number of latently infected cell 14 d after infection). Similarly, T-cell vaccination using immunodominant CD8 T-cell epitopes derived from lytic-cylce antigens decreased both acute titer and latency at d 14 after infection, and CD8 T-cells specific for the latent viral antigen decreased latency early after infection. Despite the success of these approaches in the control of acute and early latent infection, these approaches failed to produce a detectable change in long-term latency (d 28 after infection and beyond).”

These observations suggest that a decrease in viral load during the acute phase and early latent phase of the infection is not necessarily indicative of the viral load during the established latent phase.

3. Limiting Transcription Factors

a) Microcompetition

Definition

Take two polynucleotides that bind two transcription complexes. If the two complexes include the same transcription factor, the two polynucleotides will be called “microcompetitors.” A special case of microcompetition is two polynucleotides sequences that bind the same transcription complex.

Notes:

1. Transcription factors include transcription co-activators.

2. Sharing the same environment, such as cell, or chemical mix, is not required to be regarded microcompetitors. For instance, two genes which were shown once to bind the same transcription factor are regarded microcompetitors independent of their actual physical environment. To emphasize such independence, the terminology “susceptible to microcompetition” may be used.

Exemplary Assays

1. If the two polynucleotides are endogenous in a cell of interest, assay the transcription factors bound to the polynucleotides and compare the two sets of polypeptides. If the two sets include a common transcription factor, the two polynucleotides are microcompetitors.

2. In assay 1, if one or both polynucleotides are not endogenous, introduce the non endogenous polynucleotide(s) to the cell by, for instance, transfection, infection, or mutating an endogenous DNA to produce a sequence identical to the polynucleotide(s) of interest.

Notes:

1. Introduction of exogenous polynucleotides is a special case of modifying the cellular copy number of a DNA sequence. Such introduction increases the copy number from zero to a positive number. Generally, copy number may be modified by means such as the ones mentioned above, for instance, transfecting the cell with plasmids carrying a DNA sequence of interest, infecting the cell with a virus that includes the DNA sequence of interest, and mutating endogenous DNA to produce a sequence identical to the DNA sequence of interest.

2. Assume the two polynucleotides microcompete for a certain transcription factor. Assaying the copy number of at least one of the two sequences is regarded as assaying microcompetition for the transcription factor, and observing a change in the copy number of at least one of the two sequences is regarded as identification of modified microcompetition for the transcription factor.

3. Consider a transcription factor which binds a certain DNA box. Consider a specific polynucleotide that includes the DNA box, then the concentration of the factor bound to the polynucleotide is a function of the polynucleotide copy number, the concentration of the factor, and some other variables, such as the factor affinity and avidity to its box. Therefore, a change in microcompetition can be identified through observing a change in the in number of factors bound to the DNA box on the polynucleotide while all other variables are the same.

4. Note that under certain conditions, such as, fixed concentration of the factor, fixed affinity and avidity of the factor to its box on the polynucleotide, and the factor being limiting (see below), there is a “one to one” relation between the number of factors bound to its box and the copy number of the polynucleotide. Under such conditions, assaying the number of factors bound to its DNA box is regarded assaying microcompetition.

Examples

See studies in the section below entitled “Microcompetition with a limiting transcription complex.”

b) Microavailable

Definition

Consider two molecules. Assume the first molecule can take a number of different shapes. Consider one specific shape that can bind the other molecule. An increase (or decrease) in concentration of the first molecule in the shape that can bind the second molecule in the environment of the second molecule is called “increase (or decrease) in microavailability of the first molecule to the second molecule.” A shape that does not bind the second molecule is called “microunavailable to the second molecule.” Consider all the shapes of the first molecule that can bind the second molecule. Any increase (or decrease) in the concentration of all the shapes of the first molecule that can bind the second molecule is called “increase (or decrease) in microavailability of the first molecule to the second molecule.”

Notes:

1. A molecule in a complex is regarded in a different shape relative to the same molecule not in a complex, or free.

2. Consider an example of an antibody against a specific shape of the first molecule. Assume the antibody binds the first molecule in the region contacting the second molecule. Assume the antibody binds a single region of the first molecule, and that antibody binding prevents formation of a complex between the two molecules. By binding the first molecule, the antibody changes the shape of the molecule (from exposed to hidden contact region). Since the new shape does not bind the second molecule, the antibody is decreasing the microavailability of the first molecule to the second molecule. If, on the other hand, the antibody converts the first molecule to a new shape that also forms a complex with the second molecule with the same probability, microavailability is fixed.

Exemplary Assays

The following assays identify a change in microavailability following treatment.

1. Assay in a biological system (e.g., cell, cell lysate, chemical mixture) the concentrations of the first molecule in all shapes that can bind the second molecule. Apply a treatment to the system which may change the shape of the first molecule. Assay again the concentrations of the first molecule in all the shapes that can bind the second molecule. Compare the total sum of all these concentrations, before and after treatment. An increase (or decrease) in this sum indicates an increase (or decrease) in microavailability of the first molecule to the second molecule.

Examples

Antibodies specific for the first molecule may be used in immunoprecipitation, Western blot or immunoaffinity to quantify the concentrations of the first molecule before and after treatment. See also examples below.

c) Limiting Transcription Factor

Definition

Consider a transcription factor that binds a certain polynucleotide. The factor will be called “limiting in respect to the polynucleotide,” if a decrease in microavailability of the factor to the polynucleotide decreases the concentration of factor bound to the polynucleotide.

Notes:

1. The definition characterizes “limiting” by the relationship between the concentration of the microavailable factor and the concentration of the factor actually bound to the polynucleotide. According to the definition, “limiting” means a direct relationship between a decrease in microavailability of the factor and a decrease in bound factor, and “not limiting” means no such relationship between the two variables. For instance, according to this definition, a decrease in microavailable factor with no corresponding change in bound factor means “not limiting.”

2. Take the DNA sequence of a certain gene. Such DNA sequence may include coding and non coding regions of a gene, such as exons, introns, promoters, enhancers, or other segments positioned 5′ or 3′ to the coding region. Assume the transcription factor binds the DNA sequence. An assay can measure changes in DNA transcripts or mRNA instead of changes in the concentration of the bound factor. Assume the factor transactivates the gene. Since the factor is necessary for transcription, a decrease in its microavailability decreases the concentration of the factor bound to the DNA sequence, which, in turn, decreases the gene's transcription. However, an increase in concentration of factor bound to the DNA sequence does not necessarily increase transcription if binding of the factor is necessary but not sufficient for transactivation of the gene.

Exemplary Assays

1. Identify a treatment that reduces microavailability of a factor by trying different treatments, assaying microavailability following each treatment, and choosing a treatment which reduces microavailability. Assay the concentration of the factor bound to the DNA sequence in a biological system (e.g. cell of interest). Use the identified treatment to reduce microavailability. Following treatment assay again the concentration of the bound factor. A decrease in the concentration of the factor bound to the DNA sequence indicates that the factor is limiting in respect to the DNA sequence.

2. Transfect a recombinant expression vector carrying the gene expressing the transcription factor. Expression of this exogenous factor will increase the intracellular concentration of the factor. Following transfection:

(a) Assay the concentration of the factor bound to the DNA sequence. An increase in concentration of the bound factor indicates that the factor is limiting in respect to DNA sequence.

(b) Assay the gene transcription. An increase in the gene transcription indicates that the factor is limiting in respect to the gene (such an increase in transcription is expected if binding of the factor to the gene is sufficient for transactivation).

3. Contact a cell with antibodies which reduce microavailability of the factor. Following treatment:

(a) Assay the concentration of the factor bound to the DNA sequence. A decrease in concentration of the bound factor with any antibody concentration indicates that the factor is limiting in respect to sequence.

(b) Assay gene transcription. A decrease in decrease transcription with any antibody concentration indicates that the factor is limiting in respect to the gene. See Kamei 1996¹¹ which used anti-CBP immunoglubulin G (IgG). (Instead of antibodies, some studies used EIA, which, by binding to p300/cbp, also converts the shape from microavailable to microunavailable).

4. Modify the copy number of another DNA sequence, or G₂, another gene with that other DNA sequence, which also bind the factor (by, for instance, transfecting the cell with the other DNA sequence of gene, see above).

(a) Assay the concentration of the factor bound to first sequence. A decrease in concentration of the factor bound to first sequence indicates that the factor is limiting in respect to the first sequence.

(b) Assay transcription of the first gene. A decrease in transcription indicates that the factor is limiting in respect to the first gene.

Competition with the second sequence or gene, which also binds the factor, reduces the concentration of the factor bound to the first gene and, therefore, the resulting transactivation of the first gene in any concentration of the second sequence or gene. In respect to the first gene, binding of the factor to the second sequence or gene reduces microavailability of the factor to the first gene, since the factor bound to the second sequence or gene is microunavailable for binding with the first gene.

This assay is exemplified in a study reported by Kamei 1996 (ibid). The study used TPA to stimulate transcription from a promoter containing an AP-1 site. AP-1 interacts with CBP. CBP also interacts with a liganded retinoic acid receptor (RAR) and liganded glucocorticoid receptor (GR) (Kamei 1996, ibid, FIG. 1). Both RAR and GR exhibited ligand-dependent repression of TPA stimulated transcription. Induction by TPA was about 80% repressed by treatment with retinoic acid or dexamethasone. In this study, G is the gene controlled by the AP-1 promoter. In respect to this gene, the CBP·liganded-RAR complex is the microunavailable form. An increase in the concentration of CBP·liganded-RAR decreases the concentration of microavailable CBP.

In another exemplary study by Hottiger 1998¹², the two genes are HIV-CAT, which binds NF-κB, and GAL4-CAT, which binds the fusion protein GAL4-Stat2(TA). NF-κB binds p300/cbp. The GAL4-Stat2(TA) fusion protein includes the Stat2 transactivation domain which also binds p300/cbp. The study showed a close dependent inhibition of gene activation by the transactivation domain of Stat2 following transfection of a RelA expression vector (Hottiger 1998, ibid, FIG. 6A).

5. Transfect the factor and modify the copy number of the other DNA sequence or gene, which also binds the factor (by, for instance, transfecting the cell with the second sequence of gene, see also above). Following transfection:

(a) Assay concentration of the factor bound to the first sequence. Attenuated decrease in concentration of the factor bound to the first sequence indicates that F is limiting in respect to the first sequence.

(b) Assay transcription of the first gene. Attenuated decrease in transactivation caused by the second sequence or gene, indicates that the factor is limiting in respect to the first sequence (see Hottiger 1998, ibid, FIG. 6D).

6. Call the box which binds the factor the “F-box.” Transfect a cell with another DNA sequence, or gene, which carries a wild type F-box. Transfect another cell with the other DNA sequence, or gene, after mutating the F-box in the transfected sequence or gene.

(a) Assay the concentration of the factor bound to first sequence. Attenuated decrease in the concentration of the factor bound to the first sequence with the wild type but not the mutated F-box indicates that the factor is limiting in respect to the first sequence.

(b) Assay transcription of the first gene. Attenuated decrease in transactivation with the wild type but not the mutated F-box indicates that the factor is limiting in respect to the first gene.

A mutation in the F-box results in diminished binding of the factor to the second sequence or gene and an attenuated inhibitory effect on the first gene transactivation. In Kamei 1996 (ibid), mutations in the RAR AF2 domain that inhibit binding of CBP, and other coactivator proteins, abolished AP-1 repression by nuclear receptors.

8. The coactivator p300 is a 2,414-amino acid protein initially identified as a binding target of the E1A oncoprotein. cbp is a 2,441-amino acid protein initially identified as a transcriptional activator bound to phosphorylated cAMP response element (CREB) binding protein (hence, cbp). p300 and cbp share 91% sequence identity and are functionally equivalent. Both p300 and cbp are members of a family of proteins collectively referred to as p300/cbp.

Although p300/cbp is widely expressed, their cellular availability is limited. Several studies demonstrated inhibited activation of certain transcription factors resulting from competitive binding of p300/cbp to other cellular or viral proteins. For example, competitive binding of p300 or CBP to the glucocorticoid receptor (GR), or retinoic acid receptor (RAR), inhibited activation of a promoter dependent on the AP-1 transcription factor (Kamei 1996¹³). Competitive binding of cbp to STAT1α inhibited activation of a promoter dependent on both the AP-1 and ets transcription factors (Horvai 1997¹⁴). Competitive binding of p300 to STAT2 inhibited activation of a promoter dependent on the NF-κB RelA transcription factor (Hottiger 1998¹⁵). Other studies also demonstrated limited availability of p300/cbp, see, for instance, Pise-Masison 2001¹⁶, Banas 2001¹⁷, Wang 2001¹⁸, Ernst 2001¹⁹, Yuan 2001²⁰, Ghosh 2001²¹, Li 2000²², Nagarajan 2000²³, Speir 2000²⁴, Chen 2000²⁵, and Werner 2000²⁶.

d) Microcompetition for a Limiting Factor

Definition

Consider two polynucleotides that microcompete for a certain transcription factor. If the factor is limiting in respect to the two polynucleotides, the polynucleotides will be called “microcompetitors for the limiting factor.”

Exemplary Assays

1. The assays 4-7 in the section entitled “Limiting transcription factor” above, can be used to identify microcompetition for a limiting factor.

2. Modify the copy number of the two polynucleotides (by, for instance, co-transfecting recombinant vector carrying them, see also above).

(a) Assay DNA protection against enzymatic digestion (“DNase footprint assay”) of the first polynucleotide. A change in protection indicates microcompetition for a limiting factor.

(b) Assay electrophoretic gel mobility of the first polynucleotide. A change in mobility indicates microcompetition for a limiting factor.

3. If the first polynucleotide is a segment of a promoter or enhancer, or can function as a promoter or enhancer, independently, or in combination of other DNA sequences, fuse the first polynucleotide to a reporter gene such as CAT or LUC. Co-transfect the two polynucleotides. Assay for expression of the reporter gene. Specifically, assay transactivation of reporter gene following an increase in the copy number of the second polynucleotide. A change in transactivation of the reporter gene indicates microcompetition for a limiting factor.

4. A special case is when the first polynucleotide is the entire cellular genome responsible for normal cell morphology and function. Transfect the second polynucleotide and assay cell morphology and/or function (such as, binding of extracellular protein, cell replication, cellular oxidative stress, gene transcription, etc). A change in cell morphology and/or function indicates microcompetition for a limiting factor.

Notes:

1. Preferably, following co-transfection of the two polynucleotides, verify that the polynucleotides do not produce mRNA. If the sequences transcribe mRNA, block translation of proteins with, for instance, an antisense oligonucleotide specific for the exogenous MRNA. Alternatively, verify that the proteins are not involved in binding of the transcription factor to either sequence. Also, verify that co-transfection does not mutate the F-boxes in the two polynucleotides, and that the sequences do not change the methylation patterns of their F-boxes. Finally, check that the two polynucleotides do not contact each other in the F-box region.

Examples

See studies in the sections below.

4. Transcription Factors Susceptible to Microcompetition

One example of a foreign polynucleotide typically found in host cells is viral DNA. Several cellular transcription factors form complexes on viral DNA, and transactivate or suppress viral transcription. Consider GA binding protein (GABP), also called Nuclear Respiratory Factor 2 (NRF-2)²⁷, E4 Transcription factor 1 (E4TF1)²⁸, and Enhancer Factor 1A (EF-1A)²⁹, as an example. The literature lists five subunits of GABP: GABPα, GABPβ1, GABPβ2 (together called GABPβ), GABPγ1 and GABPγ2 (together called GABPγ). GABPα is an ets-related DNA-binding protein which binds the DNA motif (A/C)GGA(A/T)(G/A), termed the N-box. GABPα forms a heterocomplex with GABPβ which stimulates transcription efficiently both in vitro and in vivo. GABPα also forms a heterocomplex with GABPγ, but the heterodimer does not stimulate transcription. The degree of transactivation by GABP appears to correlate with the relative intracellular concentrations of GABPβ and GABPγ. An increase in GABPβ relative to GABPγ increases transcription, while an increase of GABPγ relative to GABPβ decreases transcription. The degree of transactivation by GABP is, therefore, a function of the ratio between GABPβ and GABPγ. Control of this ratio within the cell regulates transcription of genes with binding sites for GABP (Suzuki 1998³⁰).

The N-box is the core binding sequence of many viral enhancers including the polyomavirus enhancer area 3 (PEA3) (Asano 1990³¹), adenovirus EIA enhancer (Higashino 1993³²), Rous Sarcoma Virus (RSV) enhancer (Laimins 1984³³), Herpes Simplex Virus 1 (HSV-1) (in the promoter of the immediate early gene ICP4) (LaMarco 1989³⁴, Douville 1995³⁵), Cytomegalovirus (CMV) (IE-1 enhancer/promoter region) (Boshart 1985³⁶), Moloney Murine Leukemia Virus (Mo-MuLV) enhancer (Gunther 1994³⁷), Human Immunodeficiency Virus (HIV) (the two NF-κB binding motifs in the HIV LTR) (Flory 1996³⁸), Epstein-Barr virus (EBV) (20 copies of the N-box in the +7421/+8042 oriP/enhancer) (Rawlins 1985³⁹) and Human T-cell lymphotropic virus (HTLV) (8 N-boxes in the enhancer (Mauclere 1995⁴⁰) and one N-box in the LTR (Komfeld 1987⁴¹)). Note that some viral enhancers, for example SV40, lack a precise N-box, but still bind the GABP transcription factor (Bannert 1999⁴²).

Ample evidence exists supporting binding of GABP to the N-boxes in these viral enhancers. For instance, Flory, et al., (1996⁴³) showed binding of GABP to the HIV LTR, Douville, et al., (1995⁴⁴) showed binding of GABP to the promoter of ICP4 of HSV-1, Bruder, et al., (1991⁴⁵) and Bruder, et al., (1989⁴⁶) showed binding of GABP to the adenovirus E1A enhancer element I, Ostapchuk, et al, (1986⁴⁷) showed binding of GABP (called EF-1A in their paper) to the polyomavirus enhancer and Gunther, et al., (1994⁴⁸) showed binding of GABP to Mo-MuLV. Other studies demonstrate competition between the above viral enhancers and enhancers of other viruses. Scholer and Gruss, (1984⁴⁹) showed competition between the Moloney Sarcoma Virus (MSV) enhancer and SV40 enhancer and competition between the RSV enhancer and the BK virus enhancer.

Other cellular transcription factors also form complexes on viral DNA, and transactivate or suppress viral transcription. For instance, AML1 binds the polyomavirus (Chen 1998⁵⁰), Mo-MLV (Lewis 1999^{51,} Sun 1995⁵²), and SL3 retrovirus (Martiney 1999A⁵³, Martiney 1999B⁵⁴), NF-AT binds HIV-1 (NFAT1 binds the NF-κB site in the viral LTR) (Cron 2000⁵⁵), HNF4α binds the Hepatitis B virus (Wang 1998⁵⁶), the Smad3/Smad4 complex binds the Epstein-Barr virus (Liang 2000⁵⁷), ets1 binds the human cytomegalovirus (Chen 2000⁵⁸), NF-YB binds the human cytomegalovirus (Huang 1994⁵⁹), hepatitis B virus (Lu 1996⁶⁰, Bock 1999⁶¹), minute virus (Gu 1995⁶²), adenovirus (Song 1998⁶³), and varicella-zoster virus (Moriuchi 1995⁶⁴), ATF-2 binds the human T-cell leukemia type 1 (HTLV-1) (Xu 1996⁶⁵, Xu 1994⁶⁶), and hepatitis B virus (Choi 1997⁶⁷), p53 binds the polyomavirus (Py) (Kanda 1994⁶⁸), human CMV (Allamane 2001⁶⁹, Deb 2001⁷⁰), human immunodeficiency virus type 1 (HIV-1) (Deb 2001, ibid), and the Hepatitis B virus (Lee 1998⁷¹, Ori 1998⁷²), YY-1 binds the human papillomavirus type 18 (HPV-18) (Jundt 1995⁷³), NF-kB binds HIV (Hottiger 1998, ibid), Stat2 binds HIV (Hottiger 1998, ibid), and C/EBPβ binds the Hepatitis B virus (Lai 1999⁷⁴, Gilbert 2000⁷⁵), and HIV-1 (LTR) (Honda 1998⁷⁶), and the glucocorticoid receptor (GR) binds the mouse mammary tumor virus LTR (Pfitzner 1998, ibid).

Note that all the above mentioned transcription factors bind the limiting coactivator p300/cbp (Bannert 1999⁷⁷, Kitabayashi 1998⁷⁸, Garcia-Rodriguez 1998⁷⁹, Sisk 2000⁸⁰, Soutoglou 2000⁸¹, Janknecht 1998⁸², Feng 1998⁸³, Pouponnot 1998⁸⁴, Jayaraman 1999⁸⁵, Li 1998⁸⁶, Duyndam 1999⁸⁷, Avantaggiati 1997⁸⁸ Van Order 1999⁸⁹, Hottiger 1998⁹⁰, Gerritsen 1997⁹¹, Hottiger 1998, ibid, Paulson 1999, ibid, Gringras 1999, ibid, Bhattacharya 1996⁹², Mink 1997⁹³, Pfitzner 1998, ibid). Since p300/cbp is limiting, a transcription complex that includes p300/cbp is also limiting. For instance, since p300/cbp is limiting, GABP·p300/cbp is also limiting.

B. Aberrant Transcription and Disease

1. Introduction

Microcompetition between a foreign polynucleotide and a cellular gene for a limiting cellular transcription complex results in aberrant transcription of the cellular gene. If the limiting complex stimulates the gene transcription, microcompetition with the foreign polynucleotide reduces transcription. If the limiting complex suppresses the gene transcription, microcompetition with the foreign polynucleotide increases transcription. Aberrant transcription can result in aberrant gene expression, abnormal gene product activity, and irregular cell function. See examples in U.S. Pat. No. 7,381,526 (ibid), hereby expressly and entirely incorporated by reference and in Polansky 2003 (ibid), herby also expressly and entirely incorporated by reference.

It is a well known fact that aberrant transcription, resulting from, for instance, a mutation or hypermethylation, may result in disease. Consider, for instance, the Online Mendelian Inheritance in Man (OMIM™) database which catalogs specific mutations and their association with genetic disorders. The following examples demonstrate the effect of controlled mutation in three specific genes, MT, PDGF-B, and HSL on the subject health.

2. Examples

a) MT-I or MT-II Deficiency and Disease (Weight Gain)

Mice with mutated MT-I and MT-II genes are apparently phenotypically normal, despite reduced expression of the metallothionein genes. The disruption shows no adverse effect on their ability to reproduce and rear offspring. However, after weaning, MT-null mice consume more food and gain weight at a higher rate compared to controls. The majority of adult male mice in the MT-null colony showed moderate obesity (Beattie 1998⁹⁴). Lead treated MT-null mice showed dose-related nephromegaly, and following exposure, reduced renal function compared to wild type (Qu 2002⁹⁵). MT-I+II knock out (MTKO) mice showed higher susceptibility to autoimmune encephalomyelitis (EAE) compared to wild type (Penkowa 2001⁹⁶), and increased susceptibility to the immunosuppresseive effects of ultraviolet B radiation and cis-urocanic acid (Reeve 2000⁹⁷). MT-I/II null mice also showed increased liver and kidney damage following chronic exposure to inorganic arsenicals (Liu 2000⁹⁸).

b) PDGF-B Deficiency and Disease

In mice, a PDGF-B deficiency is embryonic lethal and is associated with cardiovascular, renal, placental and hematological disorders. Specifically, mice show formation of hemorrhage, microaneurysm, and microvessel leakage. The mice also show lack of kidney glomerular mesangial cells and microvascular pericytes, and reduced or complete loss of vascular smooth muscle cells (SMC) around small and medium sized arteries. The mice also show dilated heart and aorta, anemia and thrombocytopenia (Kaminski 2001⁹⁹, Lindahl 1997¹⁰⁰).

c) HSL Deficiency and Disease (Adipocyte Hypertrophy)

HSL knockout mice were generated by homologous recombination in embryonic stem cells. Cholesterol ester hydrolase (NCEH) activities were completely absent from both brown adipose tissue (BAT) and white adipose tissue (WAT) in mice homozygous for the mutant HSL allele (HSL−/−). The cytoplasmic area of BAT adipocytes was increased 5-fold in HSL−/− mice (Osuga 2000¹⁰¹, FIG. 3a) and the median cytoplasmic areas in WAT was enlarged 2-fold (Ibid, FIG. 3b). The HSL knockout mice showed adipocyte hypertrophy. HSL-deficient mice are normoglycemic and normoinsulinemic under basal conditions. However, after overnight fast, the mice showed reduce concentration of circulating free fatty acids (FFAs) relative to control and heterozygous mice. Moreover, an intraperitoneal glucose tolerance test of the HSL-null mice revealed insulin resistance (Roduit 2001¹⁰²). HSL-deficient male mice are infertile (Chung 2001¹⁰³). HSL-deficient mice also showed other defects associated with mobilization of triglycerides (TG), diglycerides (DG) and cholesteryl esters (Haemmerle 2002A¹⁰⁴, Haemmerle 2002B¹⁰⁵).

3. Summary

Microcompetition between a foreign polynucleotide and a cellular gene for a limiting transcription complex results in aberrant transcription of the cellular gene. Aberrant transcription results in disease. Therefore, microcompetition between a foreign polynucleotide and a cellular gene for a limiting transcription complex results in disease. When the foreign polynucleotide persists in the host cell or is latent in the host cell for an extended period of time, microcompetition between the foreign polynucleotide and the cellular gene results in a chronic disease. Chronic diseases caused by microcompetition with a foreign polynucleotide include atherosclerosis, cancer, obesity, osteoarthritis, type II diabetes, type I diabetes, multiple sclerosis, asthma, lupus, thyroiditis, inflammatory bowel disease, rheumatoid arthritis, psoriasis, atopic dermatitis, graft versus host disease, and other autoimmune diseases (see Polansky 2003, ibid).

C. Treatment with Dietary Supplements

1. Introduction

In one aspect, the invention presents methods for treating chronic diseases resulting from the presence of latent viral genomes in a host. In a preferred embodiment, the methods feature administration to a subject an effective dose of a dietary supplement to decrease the concentration of the latent viral load, prevent the load from increasing, or diminish the rate that this load is increasing. The following examples demonstrate that treatment with dietary supplements can decrease the copy number of latent viral genomes in infected cells, the number cells with latent infection, and the latent viral copy number in the cells. These examples demonstrate that dietary supplements can decrease the concentration of foreign polynucleotides in cells, and as a result increase in availability of limiting transcription complex to cellular genes, and therefore, diminish the deleterious effect of the foreign polynucleotide on transcription, cell behavior, and the host's health.

2. Treatment Protocols

a) Introduction

In one aspect, the invention presents methods for treating chronic diseases. In a preferred embodiment, the methods feature administration to a subject an effective dose of a dietary supplement that prevents or attenuates microcompetition between a foreign polynucleotide and a cellular polynucleotide or attenuates an effect of such microcompetition. For example, if the cellular polynucleotide is a GABP regulated gene, the dietary supplement can reduce the copy number of the foreign polynucleotide, stimulate the expression of the GABP regulated gene, increase the bioactivity of the GABP regulated gene, through, for instance, phosphorylation of GABP and/or increasing the bioavailability of a GABP regulated protein, through, for instance, a reduction in copy number of the foreign polynucleotides which bind GABP. A dietary supplement can also, for example, can inhibit the expression a cellular gene that increases expression as a result of microcompetition with a foreign polynucleotide, such as, tissue factor, androgen receptor, and/or inhibit replication of a p300/cbp virus.

The following sections describe standard protocols for determining effective dose, and for agent formulation for use. Additional standard protocols and background information are available in books, such as In vitro Toxicity Testing Protocols (Methods in Molecular Medicine, 43), edited by Sheila O'Hare and C K Atterwill, Humana Press, 1995; Current Protocols in Pharmacology, edited by: S J Enna, Michael Williams, John W Ferkany, Terry Kenakin, Roger D Porsolt, James P Sullivan; Current Protocols in Toxicology, edited by: Mahin Maines (Editor-in-Chief), Lucio G Costa, Donald J Reed, Shigeru Sassa, I Glenn Sipes; Remington: The Science and Practice of Pharmacy, edited by Alfonso R Gennaro, 20^{th} edition, Lippincott, Williams & Wilkins Publishers, 2000; Pharmaceutical Dosage Forms and Drug Delivery Systems, by Howard C Ansel, Loyd V Allen, Nicholas G Popovich, 7^{th} edition, Lippincott Williams & Wilkins Publishers, 1999; Pharmaceutical Calculations, by Mitchell J Stoklosa, Howard C Ansel, 10^{th} edition, Lippincott, Williams & Wilkins Publishers, 1996; Applied Biopharmaceutics and Pharmacokinetics, by Leon Shargel, Andrew B C Yu, 4^{th} edition, McGraw-Hill Professional Publishing, 1999; Oral Drug Absorption: Prediction and Assessment (Drugs and the Pharmaceutical Sciences, Vol 106), edited by Jennifer B Dressman, Hans Lennernas, Marcel Dekker, 2000; Goodman & Gilman's The Pharmacological Basis of Therapeutics, edited by Joel G Hardman, Lee E Limbird, 10^{th} edition, McGraw-Hill Professional Publishing, 2001. See also above referenced.

b) Effective Dose

Compounds can be administered to a subject, at a therapeutically effective dose, to treat, ameliorate, or prevent a chronic disease. Monitoring of patient status, using either systemic means, standard clinical laboratory assays, or assays specifically designed to monitor the bioactivity of such compounds on the foreign polynucleotide and the foreign polynucleotide's effects, can be used to establish the effective therapeutic dose and to monitor this effectiveness.

Prior to patient administration, techniques standard in the art may be used with any agent described herein to determine the LD₅₀ and ED₅₀ (lethal dose which kills one half the treated population, and effective dose in one half the population, respectively) either in cultured cells or laboratory animals. The ratio LD₅₀/ED₅₀ represents the therapeutic index which indicates the ratio between toxic and therapeutic effects. Compounds with a relatively large index are preferred. These values are also used to determine the initial therapeutic dose.

c) Formulation for Use

Those skilled in the art recognize a host of standard formulations for the agents described in this invention. For instance, the agent may be given orally by delivery in a tablet, capsule or liquid syrup. Those skilled in the art recognize pharmaceutical binding agents and carriers which protect the agent from degradation in the digestive system and facilitate uptake. Similarly, coatings for the tablet or capsule may be used to ease ingestion thereby encouraging patient compliance. If delivered in liquid suspension, additives may be included which keep the agent suspended, such as sorbitol syrup and the emulsifying agent lecithin, among others, lipophilic additives may be included, such as oily esters, or preservatives may be used to increase shelf life of the agent. Patient compliance may be further enhanced by the addition of flavors, coloring agents or sweeteners. In a related embodiment the agent may be provided in lyophilized form for reconstitution by the patient or his or her caregiver.

The agents described herein may also be delivered via buccal absorption in lozenge form. Similarly, compounds may be included in the formulation which facilitate transepithelial uptake of the agent. These include, among others, bile salts and detergents.

In every case, therapeutic agents destined for administration outside of a clinical setting may be packaged in any suitable way that assures patient compliance with regard to dose and frequency of administration.

d) Clinical Trials

Another aspect of current invention involves monitoring the effect of an agent on a treated subject in a clinical trial. In such a trial, the copy number of a foreign polynucleotide, its affinity to cellular transcription factors, the expression or bioactivity of a disrupted gene or polypeptide, or expression or bioactivity of a gene or polypeptide in a disrupted or disruptive pathway, may be used as an indicator of the agent effect on a disease state.

For example, to study the effect of a test agent in a clinical trial, blood may be collected from a subject before, and at different times following treatment with such an agent. The copy number of a foreign polynucleotide may be assayed in monocytes as described above, or the levels of expression of a disrupted gene, such as tissue factor, may be assayed by, for instance, Northern blot analysis, or RT-PCR, as described in this application, or by measuring the concentration of the protein by one of the methods described above. In this way, the copy number, or expression profile of a gene of interest or its mRNA, may serve a surrogate or direct biomarker of treatment efficacy. Accordingly, the response may be determined prior to, and at various times following agent administration. The effects of any therapeutic agent of this invention may be similarly studied if, prior to the study, a suitable surrogate or direct biomarker of efficacy, which is readily assayable, was identified.

### IV. EXAMPLES

Many dietary agents have been identified for their antiviral activities. For instance, many phytochemicals, including the flavonoids, terpenoids, organosulfur compounds, limonoids, lignans, sulphides, polyphenolics, coumarins, saponins, chlorophyllins, furyl compounds, alkaloids, polyines, thiophenes, proteins and peptides have been found to have therapeutic applications against different genetically and functionally diverse viruses. The antiviral mechanism of these agents has been explained on basis of their antioxidant activities, scavenging capacities, inhibition of DNA and RNA synthesis, inhibition of the viral entry, or inhibiting the viral reproduction etc. Large number candidate substances and their antiviral functions have been identified by a combination of in vitro and in vivo studies using different biological assays. However, no claims have been made on the capacity of these substances to decrease microcompetition between viral and cellular genes, specifically, when the virus is latent or persistent in the host. Furthermore, no claims have been made on the capacity of these substances to decrease the latent viral load, and as a result, decrease the risk developing a disease, or decrease the severity of a current disease, which is developing or was developed during the latent phase. The following section will show examples of dietary agents, includnig Glycyrrhizic acid/Glycyrrhizin, Quercetin, Epigallocatechin gallate, Cinnamon, Selenium, and Artemisinin/Artesunate, with such effects. In these examples treatment with these compounds resulted in a decrease in the concentration of a latent foreign polynucleotide in vitro and in vivo.

A. Glycyrrhizic Acid (GA)

Licorice, which has been used for thousand of years as a flavoring agent, is derived from the root of Glycyrrhiza glabra. The licorice root contains glycyrrhizic acid (GA), also called glycyrrhizin, or glycyrrhizinic acid.

1. KSHV, PEL Cells, Induced Apoptosis

The following in vitro experiments show that GA treatment induced apoptosis of primary effusion lymphoma (PEL) cells that harbor a latent Kaposi sarcoma-associated herpesvirus (KSHV)¹⁰⁶. The apoptosis decreases the number of cells harboring latent foreign polynucleotides in an infected host, decreases the latent viral load in the host, attenuates the microcompetition with cellular genes, decreases the risk of developing clinical symptoms associated with microcompetition-related diseases, and decreases the severity of already existing clinical symptoms of such diseases.

The experiments used twelve different human cell types. Five cell types, BC-1, BC-2, BC-3, BCBL-1, and BCP-1 are B-cells derived from different body cavity-based lymphomas. BC-3, BCBL-1, and BCP-1 cells harbor a latent infection with KSHV but not. BC-1 and BC-2 cells harbor both KSHV- and EBV. The examples also used primary human keratinocytes from Clonetics at the second passage. CB33 cells lymphoblastoid cells infected with EBV. Ramos cells (ATCC) are Burkitt lymphoma cells that are negative for KSHV and EBV. SLK are a KS-derived cell line negative for KSHV. KS2616 are cells prepared from a KS lesion of a HIV-negative patient. The KS2616 cells are positive for KSHV.

KSHV latent genes determine the virus persistence. Therefore, Northern blot analysis was used to measure the effect of GA treatment on the expression of three KSHV latent genes in four different KSHV-positive B cells. The there genes were the KSHV latency-associated nuclear antigen 1 (LANA-1, ORF73), the KSHV cyclin protein (v-cyclin, ORF72), and the viral FLICE-inhibitory protein (v-FLIP, K13). All KSHV-infected cells, including PEL cells, express LANA-1. This protein enables the KSHV genome to be present as an episome in latently infected cells. LANA-1 binds to p53 and inhibits the p53-induced apoptosis. LANA-1 also binds the retinoblastoma tumor-suppressor protein (Rb), which possibly inhibits the Rb-induced cell cycle arrest. V-cyclin binds to and activates the cyclin-dependent kinase 6 (cdk6), which leads to phosphorylation and inactivation of p53 and Rb.

The cells were treated with two active and nontoxic GA concentrations (3 and 4 mM). The expressions of the viral gene in treated cells were than compared to those in untreated cells. The example used BC-3 and BCBL-1 cells, which are infected with KSHV, and BC-1 and BC-2 cells, which are co-positive with KSHV and EBV.

On the basis of the growth curves, the BCBL-1 cells were treated with GA for 2 days, the BC-3 cells for 3 days, and BC-2 and BC-1 cells for 6 days. Different times were needed since the cells grow in different rates. The filters were hybridized with a probe specific for LANA-1. This probe detects the LT1 transcripts only. The results showed a dose-dependent decrease of the LT1 transcripts in n all GA-treated cells. Two additional probes that are specific for v-FLIP and v-cyclin were then hybridized. These probes detect the LT2 transcripts. The results showed a decrease in LT1 with an increase in LT2 transcripts. As control the transcript level of β-actin in the GA treated cells were assayed. The results showed similar levels of β-actin in these cells.

The decrease in LANA-1 transcripts in the GA-treated cells might suggest a change in the activity of the LT1/LT2 promoter in these cells. To determine the effect of GA treatment on the LT1/LT2 promoter, BJAB cells were transiently transfected with a reporter gene that expresses luciferase under the control of the LT1/LT2 promoter. The results showed similar level of luciferase expression in untreated and GA-treated cells. These results suggest that GA treatment does not affect the LT1/LT2 promoter activity.

Expression patterns of LANA-1, v-cyclin, and v-FLIP proteins. A Western blot and FACS analysis were used to measure the expression of LANA-1, v-cyclin, and v-FLIP proteins in of BCBL-1, BC-3, and BC-1 cells treated with GA for 2, 3, and 6 days, respectively. Untreated cells positive for KSHV show expression of LANA-1. The GA treatment of the KSHV positive cells decreased LANA-1 expression. The effect was reversible.

FACS analyses of 4 KSHV-positive B cells untreated and following treatment with GA were then used. All KSHV-positive B cells constitutively express v-cyclin. However, following treatment with GA, 35-50% of the cells over-expressed v-cyclin.

The analysis also showed similar expression of v-FLIP in GA treated and untreated cells. It has been showed that the v-FLIP protein blocks Fas-mediated apoptosis in cells latently infected with KSHV. However, in the current experiments GA treatment did not affect apoptosis, which indicates that v-FLIP although expressed, is not interfering with apoptosis.

Previous studies showed that over-expression of v-cyclin promotes apoptosis in cells with elevated levels of cdk6, and that cellular Bcl-2 or v-FLIP does not inhibit this apoptosis. Therefore, the concentration of cdk6 in the BC-1, BC-3, and BCBL-1 cells was assayed. The experiment used normal human lymphocytes, liver cells, 293 human epithelial kidney cells, and BJAB cells as controls. A Western blot analysis revealed that untreated BC-1 and BCBL-1 express high concentrations of cdk6, while untreated BC-3 cells express low concentrations of cdk5. Treatment with GA of these cells increased cdk6 expression 8- to 11-fold, which induced apoptosis in the BC-3 cells. The 293 and BJAB control cells also showed high concentrations of cdk6. However, treatment of the BJAB cells with GA did not result in apoptosis, possibly because these KSHV-negative cells show no expression of v-cyclin. The results suggest that over-expression of the v-cyclin/cdk6 complex might contribute to the apoptosis induced by the GA treatment in the KSHV-positive B cells.

The next step was to examine the biological implications of the modified latent gene expression in the KSHV-positive B cells treated with GA. One of the first intracellular changes during the onset of apoptosis is the disruption of the mitochondrial membrane potential. Proteins that are normally localized in the mitochondrial inter membrane space, such as cytochrome c and AIF, translocate to the nucleus and trigger a cascade of catabolic reactions that result in apoptosis. Following the release from the membrane, cytochrome c with Apaf-1 and procaspase-9 form the “apoptosome.” This complex activates the caspase cascade and apoptosis.

In these experiments, KSHV-positive B cells treated with GA showed the typical disruption of mitochondrial membrane, and many cells with condensed or fragmented chromatin typical of apoptosis (using a TUNEL assay). FACS analysis was used to determine the percentage of TUNEL-positive cells. The analysis showed that the GA treatment induced apoptosis in 80-95% of the KSHV-positive cells. This percentage is higher than the 35-50% of cells over-expressing v-cyclin following GA treatment, indicating that other proteins in addition to v-cyclin induce the observed apoptosis. In comparison to the KSHV-positive cells, uninfected B cells treated with GA showed no disruptions of their mitochondrial membranes or DNA condensation. Treatment with a 12-O-tetradecanoyl-phorbol-13-acetate (TPA), which is know to promote the switch from latent to lytic viral cycle, also did not disrupt the mitochondrial membranes or caused DNA condensation. The lack of the apoptotic effect of the TPA treatment indicates that lytic gene expression was not involvement in the observed apoptosis.

Usually, disruption of the mitochondrial membrane induces caspase-cascade activation and DNA fragmentation. ELISA was used to examine the activation of the caspase cascade in KSHV-positive B cells untreated and following treatment with GA. There was no activation in any sample, indicating a caspase-independent apoptosis. The experiment then targeted AIF, a mitochondrial oxidoreductase that translocates from the mitochondria to the nucleus under stress condition causing DNA loss and chromatin condensation, typical changes in apoptosis when caspases are inhibited. To identify translocation of AIF, an immunofluorescence analysis was used in KSHV-positive B cells following treatment with GA for 4 days. A cytoplasmic pattern, characteristic of mitochondrial AIF, was evident in the untreated KSHV-positive B cells and in the KSHV-negative cells (BJAB) when untreated and following treatment with. In contrast, the analysis detected a diffuse nuclear staining, indicating translocation to the nucleus, in KSHV-positive B cells following treatment with GA. These observation suggest that GA induced changes in the mitochondrial membrane potential with AIF translocation to the nucleus and DNA fragmentation only in KSHV-positive B cells. To summarize, these observations suggest that the change in the expression of the KSHV latent genes, that is, the decrease in LANA-1 expression and increase in v-cyclin expression, induces the apoptotic effects.

p53 activation and oxidative stress. Several studies showed that over-expression of the v-cyclin protein promotes cell cycle progression and apoptosis. Other studies showed that LANA-1 prevents apoptosis by inactivating p53. A decrease in p53 expression and loss of function characterizes many human malignancies. Following DNA damage, p53 undergoes phosphorylation at Ser15 or Ser20, which induces cell cycle arrest in G₁ and the initiation of DNA repair. If the cell fails to repair the damaged DNA, p53 initiates apoptosis. Therefore, the decrease of LANA-1 expression in KSHV-positive B cells might lead to p53 phosphorylation and apoptosis. To test this idea, the concentration of non-phosphorylated and phosphorylated p53 was assayed in uninfected and KSHV-positive B cells, both untreated and following treatment GA. Following treatment with GA, the experiment detected a high concentration of p53 phosphorylated at Ser15 in KSHV-positive B cells. Then BC-3 cells were transfected with the pLPCX/LANA-1 vector, a mammalian expression vector encoding LANA-1 under the control of the CMV promoter. After 24 hours, the transfected cells were treated with 4 mM GA for 4 days. A Northern blot analysis was used to confirm the presence of the 3.5-kb LANA-1 transcript. A Western blot analysis showed that GA treatment in the presence of high concentrations of LANA-1 resulted in a very low concentration of phosphorylated form of p53 and a high concentration of the non-phosphorylated form of p53. These results confirm that LANA-1 was responsible for the decrease in p53 phosphorylation. These results also support the conclusion that down regulation of LANA-1 by GA was responsible for the increase in p53 phosphorylation.

p53-induces apoptosis is associated with the formation of ROS, including H₂O₂, O₂, and OH. An increase in H₂O₂ increases the concentration of the H₂O₂ scavenger catalase. Catalase activity was assayed in KSHV-positive B cells in untreated cells and following treatment with GA. Following treatment with GA, catalase activity increased 4-fold (BC-3), 2-fold (BCBL-1), and 3-fold (BC-1) in KSHV-positive cells relative to the untreated KSHV-positive cells and relative to the KSHV-negative cells (BJAB) either untreated or following treatment with GA.

FACS was used to determine the distribution of cell cycle in KSHV-positive and uninfected B cells both untreated and following treatment with GA. After 6 days, the treatment with GA caused 99% of KSHV-positive B cells to be blocked in G₁. In contrast, only 25-50% of untreated KSHV-positive cells and uninfected cells were blocked in G₁ when untreated on following treatment with GA. These results indicate that a decrease in LANA-1 expression restores p53 function and induces cell cycle arrest of the latent KSHV-infected cells.

Summary: These results show that GA, while not being toxic at the tested levels, specifically induces apoptosis in latent KSHV-infected cells.

Note that although Curreli, et al. (2005, ibid) showed that GA induces apoptosis in cells carrying a latent infection with KSHV, they did not mention any possible influence of the apoptosis on disease, on microcompetition with foreign DNA, or on the risk of developing a microcompetition-related disease, and the severity of such disease. Specifically, they did not argue against the current misconception that latent infection does not constitute a pathogenic threat (see an expression of such misconception in Babcock 1999¹⁰⁷).

2. EBV, EA Gene, Raji Cells, Inhibition of Persistence Replication

The following in vitro experiment shows that GA treatment inhibits Epstein-Barr virus early antigen (EBV-EA) activation in latently infected cells¹⁰⁸. Since EBV-EA activation is necessary for persistent replication during the latent phase (Prang 1997¹⁰⁹), a decrease in EBV-EA transcripts decreases the latent viral load in the infected host, attenuates the microcompetition with cellular genes, decreases the risk of developing clinical symptoms associated with microcompetition-related diseases, and decreases the severity of already existing clinical symptoms of such diseases.

The example used the Raji cells, a Burkitt-lymphoma-derived cell line that harbors 50 to 60 latent, predominantly extrachromosomal, Epstein-Barr virus genomes (Adams 1987¹¹⁰). The cells were superinfected with P2HR1 (LS) virus, which results in reactivation of the latent EBV virus and replication in the superinfected cells. Following the superinfection, approximately 95% of the superinfected cells became positive for the EBV early antigen (EA). In the presence of GA, a dose-dependent inhibition of the expression of the EBV-EA was observed. The example also observed a dose-dependent inhibition of viral genome copy number determined by real-time quantitative PCR. The GA concentration required for inhibiting the EBV genome copy number and antigen expression by 50% (EC50) was approximately 5 uM.

B. Quercetin

Quercetin is a flavonoid and, or more specifically, a flavonol. It is the aglycone form of a number of other flavonoid glycosides, such as rutin and quercitrin, found in citrus fruit, buckwheat and onions. Quercetin forms the glycosides quercitrin and rutin together with rhamnose and rutinose, respectively. Quercetin is classified as IARC group 3 (no evidence of carcinogenicity in humans).

1. EBV, EA Gene, Raji Cells, Inhibition of Persistence Replication

The following in vitro experiment shows that quercetin treatment inhibits EBV-EA activation in latently infected cells¹¹¹. Since EBV-EA activation is necessary for persistent replication during the latent phase (Prang 1997, ibid), a decrease in EBV-EA transcripts decreases the latent viral load in the infected host, attenuates the microcompetition with cellular genes, decreases the risk of developing clinical symptoms associated with microcompetition-related diseases, and decreases the severity of already existing clinical symptoms of such diseases.

The experiment used Burkitt-lymphoma-derived Raji cells, which harbor 50 to 60 latent, predominantly extrachromosomal Epstein-Barr virus genomes (Adams 1987, ibid). The experiment exposed the Raji cells to EBV-EA positive serum isolated from a patient with nasopharyngeal carcinoma. The serum activated the EBV-EA. Treatment with quercetin derivatives inhibited the EBV-EA activation in the Raji cells without showing cytotoxicity. Quercetin pentaallyl ether (QPA) showed the most significant inhibitory effect on EBV-EA activation (100% inhibition at 1000 mol ratio/TPA and more than 80% inhibition at 500 mol ratio/TPA) and high viability (more than 70% viability at 1000 mol ratio/TPA).

2. HBV, cccDNA, Hep G2.2.15 Cells, Inhibition of Persistence Replication

The following in vitro experiment shows that quercetin treatment decreases the concentration of the Hepatitis B e antigen (HBeAg) in cells latently infected with the Hepatitis B virus (HBV)¹¹². Since a decrease in HBeAg concentration is associated with a decrease in the concentration of the covalently closed circular DNA (cccDNA) form, which is responsible for viral persistence during latency, a decrease in HBeAg indicates a decrease in the latent viral load in the infected host, attenuation of the microcompetition with cellular genes, a decrease in the risk of developing clinical symptoms associated with microcompetition-related diseases, and a decrease in the severity of already existing clinical symptoms of such diseases.

The experiment used the human hepatoma Hep G2.2.15 cell culture system as in vitro model to evaluate the anti-HBV effects of hyperoside, a quercetin derivative (quercetin-3-O-β-D-galactoside). The HBV-producing 2.2.15 cells were obtained from the Institute of Medicinal Biotechnology, Chinese Academy of Medical Sciences (Beijing, China). These cultures were derived from HepG2 cells that were transfected with a plasmid vector containing G418-resistance sequences and 2 head-to-tail dimmers of the HBV genome. The cells were found to produce elevated levels of HBeAg, which is expressed in HBV infected cells during the latent phase (Favre 2003¹¹³).

The experiment incubated the 2.2.15 cells for 24 hours and then treated them with different concentrations of hyperoside (0.05, 0.025, 0.0125, 0.00625, and 0.003125 g/L) in serum-free medium. The results showed that hyperoside decreased the concentration of HBeAg in the cells. The median effective concentration (IC₅₀) of hyperoside on day 4 was about 0.012 g/L, and on day 8 about 0.009 g/L.

HBV cccDNA is responsible for viral persistence during the natural course of chronic HBV infection and serves as the template for the production of HBV pregenomic RNA (pgRNA), the primary step in HBV replication. A study (Laras 2006¹¹⁴) used sensitive and specific quantitative real-time polymerase chain reaction (PCR) assays to measure the intrahepatic concentration, pgRNA production, and replicative activity of cccDNA in liver biopsy samples from 34 non-treated patients with chronic hepatitis B (CHB): 12 HBeAg(+) and 22 HBeAg(−). The results showed that in HBeAg(+) patients, the median values of cccDNA and pgRNA levels were 10-fold and 200-fold higher than in HBeAg(−), respectively. These results indicate that a decrease in HBeAg concentration is associated with a decrease in the concentration of cccDNA, and therefore, a decrease in the HBV DNA copy number during latency. Based on these results, we can conclude that hyperoside treatment decreases the copy number of latent HBV in infected cells.

C. Epigallocatechin Gallate (EGCG)

Epigallocatechin gallate (EGCG), also known as Epigallocatechin 3-gallate, is a type of catechin and is the most abundant catechin in green tea. It is the ester of epigallocatechol and gallic acid.

1. EBV, Rta Gene, P3HR1 Cells, Inhibition of Persistence Replication

The following in vitro experiment shows that EGCG treatment inhibits the activation of the EBV immediate-early protein Rta in latently infected cells¹¹⁵. Since Rta is essential to for reactivation from latency and maintenance of the latent pool (Pavlova 2003¹¹⁶), a decrease in Rta expression decreases the viral genome copy number in the latently infected cells, decreases the latent viral load in the infected host, attenuates the microcompetition with cellular genes, decreases the risk of developing clinical symptoms associated with microcompetition-related diseases, and decreases the severity of already existing clinical symptoms of such diseases.

P3HR1 is a Burkitt's lymphoma line cell line that carries a latent infection with EBV. A flow cytometry analysis that used immunostaining of Rta with primary antibody and subsequent detection of the primary antibody with fluorescein isothiocyanate (FITC) or rhodamine-conjugated secondary antibody revealed that a low percentage of untreated P3HR1 cells express the Rta protein. The analysis also revealed that following treatment with TSA, a treatment known to activate the EBV lytic cycle, the population of P3HR1 cells expressing Rta increased to 23.4%, and that treatment with 70 mM EGCG decreased the percentage of cells expressing Rta to 9.8%. Treatment with 100 mM EGCG further decreased the percentage of P3HR1 cells expressing Rta to 0.5%. To summarize: EGCG treatment significantly reduced the expression of EBV immediate-early protein Rta, which, in turn, decreases the EBV latent copy number in infected cells.

2. HBV, cccDNA, HepG2-N10 Cells, Inhibition of Persistent Replication

The following in vitro experiment shows that EGCG treatment decreases the copy number of the nuclear covalent closed circular DNA (cccDNA) form, which is characteristic of latent HBV¹¹⁷. In HBV-positive cells, the viral DNA is transported into the nucleus where it transforms into the cccDNA form. Since the cccDNA form is essential for HBV maintenance during latency, a decrease in cccDNA copy number decreases the viral genome copy number in the latently infected cells, decreases the latent viral load in the infected host, attenuates the microcompetition with cellular genes, decreases the risk of developing clinical symptoms associated with microcompetition-related diseases, and decreases the severity of already existing clinical symptoms of such diseases.

The experiment used the human hepatoblastoma cell line HepG2-N10, which was generated by transfecting HepG2 cells with a transfer plasmid which contains a 1.3 unit length of genotype A HBV genome (subtype adw2). Cells were treated with fresh medium containing various concentrations of EGCG. Treatment with a concentration of 22.9 ug/ml EGCG reduced the concentration of HBV cccDNA by 60%.

D. Cinnamaldehyde or Cinnamic Acid

Cinnamic aldehyde or cinnamaldehyde (more precisely trans-cinnamaldehyde) is the chemical compound that gives cinnamon its flavor and odor. Cinnamaldehyde occurs naturally in the bark of cinnamon trees and other species of the genus Cinnamomum like camphor and cassia. These trees are the natural source of cinnamon, and the essential oil of cinnamon bark is about 90% cinnamaldehyde. Most cinnamaldehyde is excreted in urine as cinnamic acid, an oxidized form of cinnamaldehyde.

1. EBV, EA Gene, Raji Cells, Inhibition of Persistent Replication

The following in vitro experiment shows that treatment with cinnamaldehyde or cinnamic acid inhibits EBV-EA activation in latently infected cells¹¹⁸. Since EBV-EA activation is necessary for persistent replication during the latent phase (Prang 1997, ibid), a decrease in EBV-EA transcripts decreases the latent viral load in the infected host, attenuates the microcompetition with cellular genes, decreases the risk of developing clinical symptoms associated with microcompetition-related diseases, and decreases the severity of already existing clinical symptoms of such diseases.

The experiment used the Raji cells, a Burkitt-lymphoma-derived cell line that harbors 50 to 60 latent, predominantly extrachromosomal, Epstein-Barr virus genomes (Adams 1987, ibid). The cell were treated with 2-O-tetradecanoylphorbol-13-acetate (TPA), known to promote activation of EBV-EA. The cells were treated with cinnamaldehyde or cinnamic acid and an indirect immunofluorescence technique was used to stain the EBV-EA expressing cells. The inhibition activity of the test compound was estimated by the percentage of positive cells compared to controls. The results showed that cinnamaldehyde and cinnamic acid inhibited EBV-EA activation in a dose dependent manner. The IC₅₀ of cinnamaldehyde or cinnamic acid was 158 and 40, respectively. IC₅₀ represents the mol ratio to TPA that inhibits 50% of positive controls (100%) activated with 32 pmol TPA.

E. Selenium (Se)

Selenium is a chemical element with the atomic number 34, represented by the chemical symbol Se, and an atomic mass of 78.96. Selenium is a semi metal that rarely occurs in its elemental state in nature. It is toxic in large amounts, but trace amounts of it are necessary for normal cellular function in most, if not all, animals, forming the active center of the enzymes glutathione peroxidase and thioredoxin reductase and three known deiodinase enzymes.

1. EBV, EA Gene, Raji Cells, Inhibition of Persistent Replication

The following in vitro experiment shows that treatment with Se inhibits EBV-EA activation in latently infected cells¹¹⁹. Since EBV-EA activation is necessary for persistent replication during the latent phase (Prang 1997, ibid), a decrease in EBV-EA transcripts decreases the latent viral load in the infected host, attenuates the microcompetition with cellular genes, decreases the risk of developing clinical symptoms associated with microcompetition-related diseases, and decreases the severity of already existing clinical symptoms of such diseases.

The experiment used the Raji cells, a Burkitt-lymphoma-derived cell line that harbors 50 to 60 latent, predominantly extrachromosomal, Epstein-Barr virus genomes (Adams 1987, ibid). The experiment stimulated the Raji cells with butyrate and croton oil. The stimulated cells were incubated with Se-rich rice extract. The experiment than used the indirect immunological flurescence method to count the EBV-EA positive expression rate and the inhibition rate. The results showed that Se-rich rice extract significantly inhibited the EBV-EA expression in Raji cells. At extract concentrations of 0.016, 0.078, and 0.388 μg/ml, the inhibition rate of EA expression was 2.85%, 12.88%, and 20.75%, respectively.

F. Artemisinin/Artesunate

The compound (a sesquiterpene lactone) is isolated from the plant Artemisia annua. Artesunate is a derivative of artemisinin. Artemisia has been used by Chinese herbalists for more than a thousand years in the treatment of malaria.

1. CMV, Decreased of Latent Viral Load, Transplant Patient

The following in vivo study shows that treatment with artesunate decreases the DNA viral load of CMV in a patient when initiated 120 days post infection¹²⁰. Since it takes the CMV about 4 weeks (120 days) to establish latency in the human body (Schroeder 2004¹²¹), we can conclude that the patient in this study was already at the latent phase by the time the treatment with artesunate was initiated, and that the treatment decreased the latent viral load of CMV in his body. In general, this study shows that treatment with artesunate decreases the latent viral load of foreign DNA in an infected host, and therefore, attenuates the microcompetition with cellular genes, decreases the risk of developing clinical symptoms associated with microcompetition-related diseases, and decreases the severity of already existing clinical symptoms of such diseases.

The patient in this study was a 12-year-old boy with X-linked adrenoleukodystrophy who received haploidentical T cell-depleted hematopoietic stem cells from his father. Starting from day 15 after transplantation, CMV viremia was noted. After 120 days of conventional treatments, the viral DNA load increased to 1.15*10̂6 copies/mL. At this point, oral treatment with artesunate (100 mg/day) was initiated. Results showed a favorable response with rapid reduction in viral load and improved hematopoiesis within 10 days. By day 7, the viral DNA load showed a 1.7-2.1 log decrease. The extent of the response was similar to the response the study observed during the initial ganciclovir treatment administered to the patient (1-log reduction by day 7), and to the response to ganciclovir and foscarnet treatments reported in Emery 1999¹²². Furthermore, the CMV load kinetics during artesunate treatment showed a short viral decay (T 1/2 0.9-1.9 days), similar to the kinetics of ganciclovir therapy also reported in Emery 1999 (ibid). In addition, no adverse effects were observed during the first 30 days of artesunate treatment, and no increase in viremia for 76 days after completion of therapy.

### V. PREFERRED EMBODIMENTS

The examples showed that GA, quercetin, EGCG, cinnamaldehyde or cinnamic acid, selenium, and artesunate, decrease the latent viral load in an infected host, which indicates that they can attenuate microcompetition between foreign DNA and cellular genes, and decrease the risk of developing clinical symptoms, or the severity of already existing clinical symptoms associated with such microcompetition. Therefore, a preferred embodiment of the current invention is an effective dose of one of these dietary supplements, or other dietary supplements that can serve a source for these dietary supplement, such as licorice, which can serve as a source of GA, green tea, which can serve as a source of EGCG, cinnamon, which can serve as a source of cinnamaldehyde or cinnamic acid, etc. In addition, a preferred embodiment of the current invention is any combination of these dietary supplements, for example, a capsule that include licorice, quercetin, green tea, cinnamon, and selenium.

While the above describes what are presently believed to be the preferred embodiments of the invention, those skilled in the art will realize that changes and modifications may be made thereto without departing from the spirit of the invention. It is intended to claim all such changes and modifications that fall within the true scope of the invention.

What is claimed is:

1. A method for treating an animal or human subject, the method comprising the steps of:
a. Selecting an agent, wherein said agent is a dietary supplement;
b. Administering said agent to said subject to decrease the concentration of a latent foreign polynucleotide in said subject.

2. The method in claim 1, wherein said latent foreign polynucleotide is a latent or persistent virus.

3. The method in claim 1, wherein said subject is suffering from a chronic disease, or is at risk of developing a chronic disease.

4. The method in claim 3, wherein said chronic disease is selected from the group consisting of atherosclerosis, cancer, obesity, osteoarthritis, type II diabetes, type I diabetes, multiple sclerosis, asthma, lupus, thyroiditis, inflammatory bowel disease, rheumatoid arthritis, psoriasis, atopic dermatitis, graft versus host disease, and other autoimmune diseases.

5. The method in claim 1, wherein said agent is selected from the group consisting of licorice, glycyrrhizic acid, quercetin, green tea, epigallocatechin gallate, cinnamon, cinnamaldehyde, cinnamic acid, selenium, artemisinin, and artesunate, and any combination thereof.

6. The method in claim 1, wherein said agent is consists of the combination of licorice, quercetin, green tea, cinnamon, and selenium.
